(19) United States
(12) Patent Application Publication
(10) Pub. No.: US 2023/0074331 A1
(43) Pub. Date: Mar. 9, 2023

(54) RECOMBINANT SILK SOLIDS AND FILMS

(71) Applicant: BOLT THREADS, INC., Emeryville, CA (US)

(72) Inventors: Amir Ahmad Bakhtiary Davijani, Emeryville, CA (US); William James Andrews, III, (US)

(21) Appl. No.: 17/799,051

(22) PCT Filed: Feb. 12, 2021
(86) PCT No.: PCT/US21/17871

§ 371 Date: Aug. 11, 2022

Related U.S. Application Data
(60) Provisional application No. 62/975,656, filed on Feb. 12, 2020.

(51) Int. Cl. A61K 8/64; A61K 8/02

(52) U.S. Cl. CPC A61K 8/64; A61K 8/0212

(57) ABSTRACT

The present disclosure relates to a composition for a molded body comprising a recombinant spider silk protein, and a plasticizer. Further, the present disclosure relates to a molded body comprising a recombinant spider silk protein and a plasticizer, and a process for preparing the molded body.

### CROSS REFERENCE TO RELATED APPLICATIONS

This application claims the benefit of U.S. Provisional Application No. 62/975,656, filed Feb. 12, 2020, which is hereby incorporated in its entirety by reference.

### SEQUENCE LISTING

The instant application contains a Sequence Listing which has been submitted electronically in ASCII format and is hereby incorporated by reference in its entirety. Said ASCII copy, created on Feb. 12, 2021, is named BTT-036WO_SL.txt and is 102,055 bytes in size.

### FIELD

The present disclosure relates to a composition for a molded body comprising a recombinant spider silk protein, and a plasticizer. Further, the present disclosure relates to a molded body comprising a recombinant spider silk protein and a plasticizer, and a process for preparing the molded body.

### BACKGROUND

Biorenewable and biodegradable materials are of increasing interest as an alternative to petroleum-based products. To this end, considerable effort has been made to develop methods of making materials and fibers from molecules derived from plants and animals, including recombinant silk.

However, traditional methods of processing recombinant silk, such as wet spinning, uses both solvents and coagulation baths to produce a fiber. This is disadvantageous in that the chemicals used as solvents and in coagulation baths need to be extracted from the fiber after the spinning process and subject to a closed loop process in order to provide a sustainable and responsible process. Melt spinning has also been used, but high heat can result in degradation of the recombinant silk fiber, which can negatively impact the properties of the final recombinant silk material. Furthermore, other material forms, such as solids or films, are desirable to make from recombinant silk for various applications.

What is needed therefore, are compositions of recombinant silk polypeptides, including solids and films, that have desirable mechanical and aesthetic properties, while minimizing degradation of the recombinant silk. In addition, homogeneity of the recombinant silk throughout the composition can be important. Therefore, new methods of producing such compositions are also needed.

### SUMMARY OF THE INVENTION

According to some embodiments, provided herein is a method for preparing a molded body, comprising: providing a composition comprising recombinant silk and plasticizer, wherein said composition is in a flowable state; placing said composition in a mold; applying heat and pressure to said composition in said mold; and cooling said composition to form a molded body comprising said recombinant silk.

In some embodiments, the molded body is in a solid form. In some embodiments, the molded body is a film.

In some embodiments, the recombinant silk is a recombinant silk powder distributed in said plasticizer. In some embodiments, the recombinant silk comprises a crystallinity similar to or less than the crystallinity of 18B before molding. In some embodiments, the recombinant silk protein is Nephila spider flagelliform silk or Araneus spider silk. In some embodiments, the recombinant silk is 18B. In some embodiments, the recombinant silk comprises SEQ ID NO: 1.

In some embodiments, the plasticizer is selected from the group consisting of: triethanolamine, trimethylene glycol, or propylene glycol. In some embodiments, the composition comprises 15% by weight trimethylene glycol. In some embodiments, the plasticizer is from 10-50% by weight of said composition.

In some embodiments, the heat is applied at a temperature of 130° C. In some embodiments, the pressure is applied in the range of 1,500 to 15,000 psi.

In some embodiments, the molded body has a hardness of 100 as measured by a Type A durometer. In some embodiments, the molded body has a hardness 90 or more as measured by a Type A duromoter. In some embodiments, the molded body has a hardness 50 or more, 60 or more, or 70 or more as measured by a Type D durometer. In some embodiments, the molded body can be machined, cut, or drilled and maintain its desired shape.

In some embodiments, the molded body has at least 50%, 60%, 70%, 80%, or 90% full length 18B monomers as compared to the recombinant silk of said composition in said flowable state. In some embodiments, the molded body has at least 35%, at least 40%, at least 45%, or at least 50% full length recombinant silk monomers. In some embodiments, the molded body has at least 50% total recombinant silk monomers, recombinant silk aggregates, and high molecular weight intermediates.

In some embodiments, the heat and pressure is applied for minute, 2 minutes, 3 minutes, 4 minutes, 5 minutes, 6 minutes, 8 minutes, 10 minutes, or 15 minutes. In some embodiments, the heat and pressure is applied for from 5 to 8 minutes.

In some embodiments, the method further comprises exposing said molded body to a relative humidity of at least 50% for at least 24 hours. In some embodiments, the method further comprises exposing said molded body to a relative humidity of 65% for 72 hours.

In some embodiments, the pressure is applied by a pressing load of at least 1 metric ton, at least 2 metric tons, at least at least 3 metric tons, at least 4 metric tons, or at least 5 metric tons. In some embodiments, the pressure is applied by a pressing load from 1 to 5 metric tons, or from 3 to 5 metric tons.

In some embodiments, the cooling is at a rate of about 1° C./min, about 3° C./min, or about 45° C./min.

In some embodiments, the composition has a flexural modulus of 50 MPa or more, 60 MPa or more, 70 MPa or more, 80 MPa or more, 90 MPa or more, 100 MPa or more, 150 MPa or more, 200 MPa or more, 250 MPa or more, or 300 MPa or more. In some embodiments, the composition has a maximum flexural strength of 10 MPa or more, 20 MPa or more, 30 MPa or more, 40 MPa or more, 50 MPa or more, 60 MPa or more, 70 MPa or more, 80 MPa or more MPa or more, 90 MPa or more or 100 MPa or more.

In some embodiments, the composition has an elongation percentage at break of 1 to 4%. In some embodiments, the composition has an elongation percentage at break of greater than 20%.

In some embodiments, the composition further comprises ammonium persulfate. In some embodiments, the method further comprises immersing said molded body in ammonium persulfate. In some embodiments, the molded body is cross-linked.

In some embodiments, the molded body is a cosmetic or skincare formulation.

Also provided herein is a composition comprising a recombinant silk and a plasticizer, wherein said composition is in a solid form.

In some embodiments, the molded body is in a solid form. In some embodiments, the molded body is a film.

In some embodiments, the recombinant silk is a recombinant silk powder distributed in said plasticizer. In some embodiments, the recombinant silk is 18B. In some embodiments, the recombinant silk comprises SEQ ID NO: 1.

In some embodiments, the plasticizer is selected from the group consisting of: triethanolamine, trimethylene glycol, or propylene glycol. In some embodiments, the composition comprises 15% by weight trimethylene glycol. In some embodiments, the plasticizer is from 10-50% by weight of said composition.

In some embodiments, the molded body has a hardness of 100 as measured by a Type A durometer. In some embodiments, the molded body has a hardness 90 or more as measured by a Type A duromoter. In some embodiments, the molded body has a hardness 50 or more, 60 or more, or 70 or more as measured by a Type D durometer. In some embodiments, the molded body can be machined, cut, or drilled and maintain its desired shape.

In some embodiments, the molded body has at least 50%, 60%, 70%, 80%, or 90% full length 18B monomers as compared to the recombinant silk of said composition in said flowable state. In some embodiments, the molded body has at least 35%, at least 40%, at least 45%, or at least 50% full length recombinant silk monomers. In some embodiments, the molded body has at least 50% total recombinant silk monomers, recombinant silk aggregates, and high molecular weight intermediates.

In some embodiments, the composition has a flexural modulus of 50 MPa or more, 60 MPa or more, 70 MPa or more, 80 MPa or more, 90 MPa or more, 100 MPa or more, 150 MPa or more, 200 MPa or more, 250 MPa or more, or 300 MPa or more. In some embodiments, the composition has a maximum flexural strength of 10 MPa or more, 20 MPa or more, 30 MPa or more, 40 MPa or more, 50 MPa or more, 60 MPa or more, 70 MPa or more, 80 MPa or more MPa or more, 90 MPa or more or 100 MPa or more.

In some embodiments, the composition has an elongation percentage at break of 1 to 4%. In some embodiments, the composition has an elongation percentage at break of greater than 20%.

In some embodiments, the composition further comprises ammonium persulfate. In some embodiments, the molded body is cross-linked.

In some embodiments, the molded body is a cosmetic or skincare formulation.

### BRIEF DESCRIPTION OF THE DRAWINGS

The foregoing and other objects, features and advantages will be apparent from the following description of particular embodiments of the invention, as illustrated in the accompanying drawings.

FIG. 1 shows an image of additional solvent pressed out from the plasticized powder during pressing.

FIG. 2 illustrates pressed solids (i.e., molded bodies) with trimethylene glycol.

FIG. 3 shows a picture of pressed solids indicating darkening of the protein color over time.

FIG. 4A, FIG. 4B, and FIG. 4C show an analysis of temperature as a function of time. (FIG. 4A) Slow cooling of solid within the mold yields cooling rate of 0.92° C./min (FIG. 4B) Medium cooling of solid in ambient air resting outside of mold yields cooling rate of 2.7° C./min (FIG. 4C) Fast cooling of solid outside of mold in dry ice yields cooling rate of 45.2° C./min.

FIG. 5 shows Force vs Distance curves to assess the effect of conditioning at 65% RH for a minimum of 72 hours on the mechanical properties of 18B solids. Series 1, 3, 5, 7, and 9 are conditioned and series 2, 4, 6, 8, and 11 are not conditioned.

FIG. 6 shows the morphology of solids subjected to 1-minute hold time (L) conditioned for 72 hours in 65% RH environment and (R) unconditioned. Comparable particle sizes, though the conditioned specimen has more clearly amorphous regions between particles possibly lending to increased ductility.

FIG. 7 shows Force vs Distance curves to assess the effect of cooling rate on the mechanical properties of 18B solids. The 10, 11, and 12 series correspond to slow, medium, and fast cooling rates, respectively.

FIG. 8 shows recombinant silk molded body comparisons between (A) slow cool (B) medium cool and (C) fast cool.

FIG. 9 shows Force vs Distance curves to assess the effect of average load on the mechanical properties of 18B solids. The 13, 14, 15, 16, and 17 series correspond to 1, 2, 3, 4, and 5 metric tons, respectively.

FIG. 10 shows an image of a recombinant silk molded body with porosity voids on solids surface. Visible voids on surface of many solids surfaces on left side of image. Right side shows dispersed protein particles.

FIG. 11 shows the effect of average pressing load on the recombinant silk molded body. Decrease in amount of dispersed protein particles as average load increases from (A) 1 metric ton to (B) 3 metric tons to (C) 5 metric tons.

FIG. 12 shows Force vs Distance curves to assess the effect of mold time on the mechanical properties of 18B solids. Series 2, 4, 6, 8, 11, 18, 19, 20, and 21 correspond to 1 minute, 2 minutes, 3 minutes, 4 minutes, 5 minutes, 6 minutes, 8 minutes, 10 minutes, and 15 minutes, respectively.

FIG. 13 shows average flexural modulus (MPa) over holding time for recombinant silk molded bodies. Error bars show sample standard deviation.

FIG. 14 shows average flexural strength (MPa) over holding time for recombinant silk molded bodies. Error bars show sample standard deviation.

FIG. 15 shows average elongation at break (%) over holding time for recombinant silk molded bodies. Error bars show sample standard deviation.

FIG. 16 shows the effect of mold time on the morphology of unconditioned recombinant silk molded bodies subjected to various hold times maintaining equal average load and cooling rate: (A) 1 minute (B) 3 minutes (C) 5 minutes (D) 8 minutes (E) 10 minutes (F) 15 minutes.

FIG. 17 shows the effect of mold time on the morphology of unconditioned recombinant silk molded bodies subjected to 1-minute hold vs 5-minute hold. Macroscopic visual examination between 1-minute hold time and 5-minute hold time against (A) solid black surface (B, C) bright light. Longer hold times have fewer noticeable powder clumps and are more translucent.

FIG. 18 shows a post-fracture surface of a recombinant silk molded body imaged with Benchtop SEM. Imaging of surface across different hold times. (A) 1-minute hold time darkened for greater contrast (B) 5-minute hold time (C) 15-minute hold time.

FIG. 19 shows a cross-linked 18B/TEOA sample of a recombinant silk molded body.

FIG. 20A and FIG. 20B show APS cross-linked 18B/glycerol films dry (FIG. 20A), or after leaving in water for 1 hour (FIG. 20B). The left film was soaked in the cross-linking solution for 10 minutes, while the right film was soaked for 1 hour.

FIG. 21 shows cross-linked 18B solid frames using glutaraldehyde chemistry placed in water container did not show any structural changes within 30 minutes testing time.

FIG. 22 shows 18B/glycerol powder dispersed on surface

FIG. 23 shows the transparency and drapability of recombinant silk/glycerol films.

FIG. 24 shows an example of a laser cut recombinant silk/glycerol film.

FIG. 25 shows an image of 18B powder without plasticizer pressed at 130° C.

FIG. 26 shows the formation of flash during press molding.

FIG. 27 shows an image of a molded 18B solid prepared by pressing with 1,3 propanediol (left) and an image of the solid reprocessed and pressed at 130° C. to form a thin film (right).

### DETAILED DESCRIPTION

The details of various embodiments of the invention are set forth in the description below. Other features, objects, and advantages of the invention will be apparent from the description. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

### Definitions

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and claims, the word “comprise” or variations such as “comprises” or “comprising,” will be understood to imply the inclusion of a stated integer or group of integers but not the exclusion of any other integer or group of integers.

The term “molded body” or “solid” as defined herein refer to a body manufactured by shaping liquid or pliable raw material using a rigid frame called a mold, such as the molding process including but not limited to extrusion molding, injection molding, compression molding, blow molding, laminating, matrix molding, rotational molding, spin casting, transfer molding, thermoforming, and the like.

The term “glass transition” as used herein refers to the transition of a substance or composition from a hard, rigid or “glassy” state into a more pliable, “rubbery” or “viscous” state.

The term “glass transition temperature” as used herein refers to the temperature at which a substance or composition undergoes a glass transition.

The term “melt transition” as used herein refers to the transition of a substance or composition from a rubbery state to a less-ordered liquid phase or flowable state.

The term “melting temperature” as used herein refers to the temperature range over which a substance undergoes a melt transition.

The term “plasticizer” as used herein refers to any molecule that interacts with a polypeptide sequence to prevent the polypeptide sequence from forming tertiary structures and bonds and/or increases the mobility of the polypeptide sequence.

The term “flowable state” as used herein refers to a composition that has characteristics that are substantially the same as liquid (i.e. has transitioned from a rubbery state into a more liquid state).

The term “crosslinked” or “cross-linked” as used herein refers to a bond formed between a reactive group on two or more proteins. Cross-linking can be performed, e.g., by enzymatic cross-linking or photo cross-linking. For example, ammonium persulfate and light or ammonium persulfate and heat can be used to cross-link silk or silk-like polypeptides.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Overview

Provided herein is a composition for a molded body, comprising a recombinant spider silk protein and a plasticizer, wherein the composition comprises desirable mechanical properties, such as strength, flexibility, stiffness. In addition, in some embodiments the composition is homogeneous or substantially homogeneous in a melted or flowable state. Also, in some embodiments, the recombinant spider silk protein is substantially non-degraded after it is formed into a molded body (e.g. degraded in an amount of less than 10%, or often less than 6% by weight). In preferred embodiments, the recombinant silk protein comes in the form of powder. Also provided herein are methods of generating such compositions, including placing a composition comprising a silk protein and a plasticizer in to a mold, and forming a molded body by applying pressure and heat to the composition in the mold, followed by cooling the molded body and optionally exposing to additional conditioning, such as high relative humidity. In preferred embodiments, the heat is low enough such that the heat and time of molding are low enough such that there is minimal degradation of the recombinant silk protein in the molded body to maintain desirable properties that arise from the use of recombinant silk.

### Recombinant Silk Proteins

The present disclosure describes embodiments of the invention including molded bodies, such as solids and films, synthesized from synthetic proteinaceous copolymers (i.e., recombinant polypeptides), such as silk or silk-like recombinant polypeptides. In some embodiments, the molded bodies, such as solids or films, form a cosmetic or skincare formulation (e.g., solutions applied to the skin or hair). The molded bodies provided herein may contain various humectants, emollients, occlusive agents, active agents and cosmetic adjuvants, depending on the embodiment and the desire efficacy of the formulation.

Suitable proteinaceous co-polymers are discussed in U.S. Patent Publication No. 2016/0222174, published Aug. 45, 2016, U.S. Patent Publication No. 2018/0111970, published Apr. 26, 2018, and U.S. Patent Publication No. 2018/0057548, published Mar. 1, 2018, each of which are incorporated by reference herein in its entirety. In addition, proteinaceous co-polymers having a crystallinity similar to or less than 18B and/or similar extensibility index (e.g., Nephila spider flagelliform silk, Araneus spider silk, regenerated silk fibroin) are suitable to be used in the molded bodies described herein. In some embodiments, other non-silk proteins with similar properties suitable for forming molded bodies, such as titin protein, are suitable proteinaceous co-polymers for forming molded bodies as described herein.

In some embodiments, the synthetic proteinaceous copolymers are made from silk-like polypeptide sequences. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful molded body compositions by secretion from an industrially scalable microorganism. Large (approximately 40 kDa to approximately 100 kDa) block copolymer polypeptides engineered from silk repeat domain fragments, including sequences from almost all published amino acid sequences of spider silk polypeptides, can be expressed in the modified microorganisms described herein. In some embodiments, silk polypeptide sequences are matched and designed to produce highly expressed and secreted polypeptides capable of molded body formation.

In some embodiments, block copolymers are engineered from a combinatorial mix of silk polypeptide domains across the silk polypeptide sequence space. In some embodiments, the block copolymers are made by expressing and secreting in scalable organisms (e.g., yeast, fungi, and gram positive bacteria). In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is >100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent molded bodies that have properties that recapitulate the properties of corresponding molded bodies made from natural silk polypeptides.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of molded body formation.

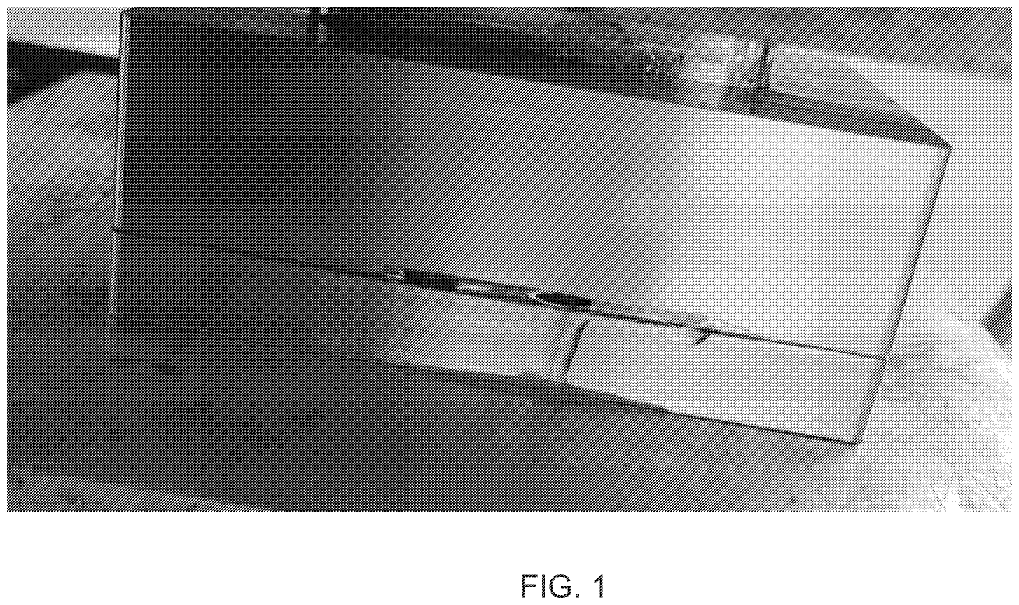

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture, as depicted in FIG. 1. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 3) is, for the purposes of the present invention, the same as GSGAG (SEQ ID NO: 4) and the same as GGSGA (SEQ ID NO: 5); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1
Samples of Block Sequences

| Species | Silk Type | Representative Block Amino Acid Sequence |
|---|---|---|
| Aliatypus gulosus | Fibroin 1 | GAASSSSTIITTKSASASAAADASAAATASAASRSSANAAASAFAQS |
| | | FSSILLESGYFCSIFGSSISSSYAAAIASAASRAAAESNGYTTHAYA |
| | | CAKAVASAVERVTSGADAYAYAQAISDALSHALLYTGRLNTANANSL |
| | | ASAFAYAFANAAAQASASSASAGAASASGAASASGAGSAS (SEQ |
| | | ID NO: 6) |
| Plectreurys tristis | Fibroin 1 | GAGAGAGAGAGAGAGAGSGASTSVSTSSSSGSGAGAGAGSGAGSGAG |
| | | AGSGAGAGAGAGGAGAGFGSGLGLGYGVGLSSAQAQAQAQAAAQAQA |
| | | QAQAQAYAAAQAQAQAQAQAQAAAAAAAAAAA (SEQ ID NO: 7) |
| Plectreurys tristis | Fibroin 4 | GAAQKQPSGESSVATASAAATSVTSGGAPVGKPGVPAPIFYPQGPLQ |
| | | QGPAPGPSNVQPGTSQQGPIGGVGGSNAFSSSFASALSLNRGFTEVI |
| | | SSASATAVASAFQKGLAPYGTAFALSAASAAADAYNSIGSGANAFAY |
| | | AQAFARVLYPLVQQYGLSSSAKASAFASAIASSFSSGTSGQGPSIGQ |
| | | QQPPVTISAASASAGASAAAVGGGQVGQGPYGGQQQSTAASASAAAA |
| | | TATS (SEQ ID NO: 8) |
| Araneus | TuSp | GNVGYQLGLKVANSLGLGNAQALASSLSQAVSAVGVGASSNAYANAV |
| gemmoides | | SNAVGQVLAGQGILNAANAGSLASSFASALSSSAASVASQSASQSQA |
| | | ASQSQAAASAFRQAASQSASQSDSRAGSQSSTKTTSTSTSGSQADSR |
| | | SASSSASQASASAFAQQSSASLSSSSSFSSAFSSATSISAV (SEQ |
| | | ID NO: 9) |
| Argiope aurantia | TuSp | GSLASSFASALSASAASVASSAAAQAASQSQAAASAFSRAASQSASQ |
| | | SAARSGAQSISTTTTTSTAGSQAASQSASSAASQASASSFARASSAS |
| | | LAASSSFSSAFSSANSLSALGNVGYQLGFNVANNLGIGNAAGLGNAL |
| | | SQAVSSVGVGASSSTYANAVSNAVGQFLAGQGILNAANA (SEQ ID |
| | | NO: 10) |
| Deinopis spinosa | TuSp | GASASAYASAISNAVGPYLYGLGLFNQANAASFASSFASAVSSAVAS |
| | | ASASAASSAYAQSAAAQAQAASSAFSQAAAQSAAAASAGASAGAGAS |
| | | AGAGAVAGAGAVAGAGAVAGASAAAASQAAASSSASAVASAFAQSAS |
| | | YALASSSAFANAFASATSAGYLGSLAYQLGLTTAYNLGLSNAQAFAS |
| | | TLSQAVTGVGL (SEQ ID NO: 11) |
| Nephila clavipes | TuSp | GATAASYGNALSTAAAQFFATAGLLNAGNASALASSFARAFSASAES |
| | | QSFAQSQAFQQASAFQQAASRSASQSAAEAGSTSSSTTTTTSAARSQ |
| | | AASQSASSSYSSAFAQAASSSLATSSALSRAFSSVSSASAASSLAYS |
| | | IGLSAARSLGIADAAGLAGVLARAAGALGQ (SEQ ID NO: 12) |
| Argiope trifasciata | Flag | GGAPGGGPGGAGPGGAGFGPGGGAGFGPGGGAGFGPGGAAGGPGGPG |
| | | GPGGPGGAGGYGPGGAGGYGPGGVGPGGAGGYGPGGAGGYGPGGSGP |
| | | GGAGPGGAGGEGPVTVDVDVTVGPEGVGGGPGGAGPGGAGFGPGGGA |
| | | GFGPGGAPGAPGGPGGPGGPGGPGGPGGVGPGGAGGYGPGGAGGVGP |
| | | AGTGGFGPGGAGGFGPGGAGGFGPGGAGGFGPAGAGGYGPGGVGPGG |
| | | AGGFGPGGVGPGGSGPGGAGGEGPVTVDVDVSV (SEQ ID NO: |
| | | 13) |
| Nephila clavipes | Flag | GVSYGPGGAGGPYGPGGPYGPGGEGPGGAGGPYGPGGVGPGGSGPGG |
| | | YGPGGAGPGGYGPGGSGPGGYGPGGSGPGGYGPGGSGPGGYGPGGSG |
| | | PGGYGPGGYGPGGSGPGGSGPGGSGPGGYGPGGTGPGGSGPGGYGPG |
| | | GSGPGGSGPGGYGPGGSGPGGFGPGGSGPGGYGPGGSGPGGAGPGGV |
| | | GPGGFGPGGAGPGGAAPGGAGPGGAGPGGAGPGGAGPGGAGPGGAGP |
| | | GGAGGAGGAGGSGGAGGSGGTTIIEDLDITIDGADGPITISEELPIS |
| | | GAGGSGPGGAGPGGVGPGGSGPGGVGPGGSGPGGVGPGGSGPGGVGP |
| | | GGAGGPYGPGGSGPGGAGGAGGPGGAYGPGGSYGPGGSGGPGGAGGP |
| | | YGPGGEGPGGAGGPYGPGGAGGPYGPGGAGGPYGPGGEGGPYGP |
| | | (SEQ ID NO: 14) |
| Latrodectus | AcSp | GINVDSDIGSVTSLILSGSTLQMTIPAGGDDLSGGYPGGFPAGAQPS |
| hesperus | | GGAPVDFGGPSAGGDVAAKLARSLASTLASSGVFRAAFNSRVSTPVA |
| | | VQLTDALVQKIASNLGLDYATASKLRKASQAVSKVRMGSDTNAYALA |
| | | ISSALAEVLSSSGKVADANINQIAPQLASGIVLGVSTTAPQFGVDLS |
| | | SINVNLDISNVARNMQASIQGGPAPITAEGPDFGAGYPGGAPTDLSG |
| | | LDMGAPSDGSRGGDATAKLLQALVPALLKSDVFRAIYKRGTRKQVVQ |
| | | YVTNSALQQAASSLGLDASTISQLQTKATQALSSVSADSDSTAYAKA |
| | | FGLAIAQVLGTSGQVNDANVNQIGAKLATGILRGSSAVAPRLGIDLS |
| | | (SEQ ID NO: 15) |
| Argiope trifasciata | AcSp | GAGYTGPSGPSTGPSGYPGPLGGGAPFGQSGFGGSAGPQGGFGATGG |
| | | ASAGLISRVANALANTSTLRTVLRTGVSQQIASSWQRAAQSLASTL |
| | | GVDGNNLARFAVQAVSRLPAGSDTSAYAQAFSSALFNAGVLNASNID |
| | | TLGSRVLSALLNGVSSAAQGLGINVDSGSVQSDISSSSSFLSTSSSS |
| | | ASYSQASASSTS (SEQ ID NO: 16) |
| Uloborus diversus | AcSp | GASAADIATAIAASVATSLQSNGVLTASNVSQLSNQLASYVSSGLSS |
| | | TASSLGIQLGASLGAGFGASAGLSASTDISSSVEATSASTLSSSASS |
| | | TSVVSSINAQLVPALAQTAVLNAAFSNINTQNAIRIAELLTQQVGRQ |
| | | YGLSGSDVATASSQIRSALYSVQQGSASSAYVSAIVGPLITALSSRG |
| | | VVNASNSSQIASSLATAILQFTANVAPQFGISIPTSAVQSDLSTISQ |
| | | SLTAISSQTSSSVDSSTSAFGGISGPSGPSPYGPQPSGPTFGPGPSL |
| | | SGLTGFTATFASSFKSTLASSTQFQLIAQSNLDVQTRSSLISKVLIN |
| | | ALSSLGISASVASSIAASSSQSLLSVSA (SEQ ID NO: 17) |
| Euprosthenops | MaSp1 | GGQGGQGQGRYGQGAGSSAAAAAAAAAAAAAA (SEQ ID NO: |
| australis | | 18) |
| Tetragnatha | MaSp1 | GGLGGGQGAGQGGQQGAGQGGYGSGLGGAGQGASAAAAAAAA (SEQ |
| kauaiensis | | ID NO: 19) |
| Argiope aurantia | MaSp2 | GGYGPGAGQQGPGSQGPGSGGQQGPGGLGPYGPSAAAAAAAA (SEQ |
| | | ID NO: 20) |
| Deinopis spinosa | MaSp2 | GPGGYGGPGQQGPGQGQYGPGTGQQGQGPSGQQGPAGAAAAAAAAA |
| | | (SEQ ID NO: 21) |
| Nephila clavata | MaSp2 | GPGGYGLGQQGPGQQGPGQQGPAGYGPSGLSGPGGAAAAAAA (SEQ |
| | | ID NO: 22) |
| Deinopis Spinosa | MiSp | GAGYGAGAGAGGGAGAGTGYGGGAGYGTGSGAGYGAGVGYGAGAGAG |
| | | GGAGAGAGGGTGAGAGGGAGAGYGAGTGYGAGAGAGGGAGAGAGAGA |
| | | GAGAGAGSGAGAGYGAGAGYGAGAGAGGVAGAGAAGGAGAAGGAGAA |
| | | GGAGAAGGAGAGAGAGSGAGAGAGGGARAGAGG (SEQ ID NO: |
| | | 23) |
| Latrodectus | MiSp | GGGYGRGQGAGAGVGAGAGAAAGAAAIARAGGYGQGAGGYGQGQGAG |
| hesperus | | AAAGAAAGAGAGGYGQGAGGYGRGQGAGAGAGAGAGARGYGQGAGAG |
| | | AAAGAAASAGAGGYGQGAGGYGQGQGAGAAAGAAASAGAGGYGQGAG |
| | | GYGQGQGA (SEQ ID NO: 24) |
| Nephila clavipes | MiSp | GAGAGGAGYGRGAGAGAGAAAGAGAGAAAGAGAGAGGYGGQGGYGAG |
| | | AGAGAAAAAGAGAGGAAGYSRGGRAGAAGAGAGAAAGAGAGAGGYGG |
| | | QGGYGAGAGAGAAAAAGAGSGGAGGYGRGAGAGAAAGAGAAAGAGAG |
| | | AGGYGGQGGYGAGAGAAAAA (SEQ ID NO: 25) |
| Nephilengys | MiSp | GAGAGVGGAGGYGSGAGAGAGAGAGAASGAAAGAAAGAGAGGAGGYG |
| cruentata | | TGQGYGAGAGAGAGAGAGGAGGYGRGAGAGAGAGAGGAGGYGAGQGY |
| | | GAGAGAGAAAAAGDGAGAGGAGGYGRGAGAGAGAGAAAGAGAGGAGG |
| | | YGAGQGYGAGAGAGAAAGAGAGGAGGYGAGQGYGAGAGAGAAAAA |
| | | (SEQ ID NO: 26) |
| Uloborus diversus | MiSp | GSGAGAGSGYGAGAGAGAGSGYGAGSSASAGSAINTQTVTSSTTTSS |
| | | QSSAAATGAGYGTGAGTGASAGAAASGAGAGYGGQAGYGQGAGASAR |
| | | AAGSGYGAGAGAAAAAGSGYGAGAGAGAGSGYGAGAAA (SEQ ID |
| | | NO: 27) |
| Uloborus diversus | MiSp | GAGAGYRGQAGYIQGAGASAGAAAAGAGVGYGGQAGYGQGAGASAGA |
| | | AAAAGAGAGRQAGYGQGAGASAGAAAAGAGAGRQAGYGQGAGASAGA |
| | | AAAGADAGYGGQAGYGQGAGASAGAAASGAGAGYGGQAGYGQGAGAS |
| | | AGAAAAGAGAGYLGQAGYGQGAGASAGAAAGAGAGYGGQAGYGQGTG |
| | | AAASAAASSA (SEQ ID NO: 28) |
| Araneus | MaSp1 | GGQGGQGGYGGLGSQGAGQGGYGAGQGAAAAAAAAGGAGGAGRGGLG |
| ventricosus | | AGGAGQGYGAGLGGQGGAGQAAAAAAAGGAGGARQGGLGAGGAGQGY |
| | | GAGLGGQGGAGQGGAAAAAAAAGGQGGQGGYGGLGSQGAGQGGYGAG |
| | | QGGAAAAAAAAGGQGGQGGYGGLGSQGAGQGGYGGRQGGAGAAAAAA |
| | | AA (SEQ ID NO: 29) |
| Dolomedes | MaSp1 | GGAGAGQGSYGGQGGYGQGGAGAATATAAAAGGAGSGQGGYGGQGGL |
| tenebrosus | | GGYGQGAGAGAAAAAAAAAGGAGAGQGGYGGQGGQGGYGQGAGAGAA |
| | | AAAAGGAGAGQGGYGGQGGYGQGGGAGAAAAAAAASGGSGSGQGGYG |
| | | GQGGLGGYGQGAGAGAGAAASAAAA (SEQ ID NO: 30) |
| Nephilengys | MaSp | GGAGQGGYGGLGGQGAGAAAAAAGGAGQGGYGGQGAGQGAAAAAASG |
| cruentata | | AGQGGYEGPGAGQGAGAAAAAAGGAGQGGYGGLGGQGAGQGAGAAAA |
| | | AAGGAGQGGYGGLGGQGAGQGAGAAAAAAGGAGQGGYGGQGAGQGAA |
| | | AAAAGGAGQGGYGGLGSGQGGYGRQGAGAAAAAAAA (SEQ ID |
| | | NO: 31) |
| Nephilengys | MaSp | GGAGQGGYGGLGGQGAGAAAAAAGGAGQGGYGGQGAGQGAAAAAASG |
| cruentata | | AGQGGYGGPGAGQGAGAAAAAAGGAGQGGYGGLGGQGAGQGAGAAAA |
| | | AAGGAGQGGYGGQGAGQGAAAAAAGGAGQGGYGGLGSGQGGYGGQGA |
| | | GAAAAAGGAGQGGYGGLGGQGAGQGAGAAAAAA (SEQ ID NO: |
| | | 32) |

Molded body-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers or molded bodies include natural amino acid sequence information and other modifications described herein. The repeat domain is decomposed into repeat sequences containing representative blocks, usually 1-8 depending upon the type of silk, that capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3×FLAG nucleotide coding sequence. In some embodiments, the silk polypeptide nucleotide coding sequence is operatively linked to other affinity tags such as 6-8 His residues (SEQ ID NO: 33).

In some embodiments, the recombinant spider silk polypeptides are based on recombinant spider silk protein fragment sequences derived from MaSp2, such as from the species Argiope bruennichi. In some embodiments, the molded body contains protein molecules that include two to twenty repeat units, in which a molecular weight of each repeat unit is greater than about 20 kDa. Within each repeat unit of the copolymer are more than about 60 amino acid residues, often in the range 60 to 100 amino acids that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms molded bodies with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the spider silk protein comprises: at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; a glycine-rich region with 12 or more consecutive amino acids, comprising a glycine content of at least 40% and an alanine content of less than 30%.

In some embodiments, wherein the recombinant spider silk protein comprises repeat units wherein each repeat unit has at least 95% sequence identity to a sequence that comprises from 2 to 20 quasi-repeat units; each quasi-repeat unit comprises {GGY-[GPG-X₁]ₙ₁-GPS-(A)ₙ₂} (SEQ ID NO: 34), wherein for each quasi-repeat unit; X₁ is independently selected from the group consisting of SGGQQ (SEQ ID NO: 35), GAGQQ (SEQ ID NO: 36), GQGPY (SEQ ID NO: 37), AGQQ (SEQ ID NO: 38), and SQ; and n1 is from 4 to 8, and n2 is from 6-10. The repeat unit is composed of multiple quasi-repeat units.

In some embodiments, 3 “long” quasi repeats are followed by 3 “short” quasi-repeat units. As mentioned above, short quasi-repeat units are those in which n1=4 or 5. Long quasi-repeat units are defined as those in which n1=6, 7 or 8. In some embodiments, all of the short quasi-repeats have the same X₁ motifs in the same positions within each quasi-repeat unit of a repeat unit. In some embodiments, no more than 3 quasi-repeat units out of 6 share the same X₁ motifs.

In additional embodiments, a repeat unit is composed of quasi-repeat units that do not use the same X₁ more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X₁ more than 2 times in a single quasi-repeat unit of the repeat unit.

In some embodiments, the recombinant spider silk polypeptide comprises the polypeptide sequence of SEQ ID NO: 1 (i.e., 18B). In some embodiments, the repeat unit is a polypeptide comprising SEQ ID NO: 2. These sequences are provided in Table 2:

TABLE 2
Exemplary polypeptides sequences of recombinant protein and
repeat unit

| SEQ ID | Polypeptide Sequence |
|---|---|
| SEQ ID | GGYGPGAGQQGPGSGGQQGPGGQGPYGSGQQGPGGAGQQGPGGQGPYGPGAAAAAAAAAG |
| NO: 1 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAAGGYGPGAGQQGPGSGGQQGPGGQGPYGSGQQGPGGAGQQGPGGQG |
| | PYGPGAAAAAAAAAGGYGPGAGQQGPGGAGQQGPGSQGPGGQGPYGPGAGQQGPGSQGPG |
| | SGGQQGPGGQGPYGPSAAAAAAAAAGGYGPGAGQRSQGPGGQGPYGPGAGQQGPGSQGPG |
| | SGGQQGPGGQGPYGPSAAAAAAAAGGYGPGAGQQGPGSQGPGSGGQQGPGGQGPYGPGAA |
| | AAAAAVGGYGPGAGQQGPGSQGPGSGGQQGPGGQGPYGPSAAAAAAAAGGYGPGAGQQGP |
| | GSQGPGSGGQQGPGGQGPYGPSAAAAAAAAGGYGPGAGQQGPGSGGQQGPGGQGPYGSGQ |
| | QGPGGAGQQGPGGQGPYGPGAAAAAAAAAGGYGPGAGQQGPGGAGQQGPGSQGPGGQGPY |
| | GPGAGQQGPGSQGPGSGGQQGPGGQGPYGPSAAAAAAAAAGGYGPGAGQRSQGPGGQGPY |
| | GPGAGQQGPGSQGPGSGGQQGPGGQGPYGPSAAAAAAAAGGYGPGAGQQGPGSQGPGSGG |
| | QQGPGGQGPYGPGAAAAAAAVGGYGPGAGQQGPGSQGPGSGGQQGPGGQGPYGPSAAAAA |
| | AAAGGYGPGAGQQGPGSQGPGSGGQQGPGGQGPYGPSAAAAAAAA |
| SEQ ID | GGYGPGAGQQGPGSGGQQGPGGQGPYGSGQQGPGGAGQQGPGGQGPYGPGAAAAAAAAAG |
| NO: 2 | GYGPGAGQQGPGGAGQQGPGSQGPGGQGPYGPGAGQQGPGSQGPGSGGQQGPGGQGPYGP |
| | SAAAAAAAAAGGYGPGAGQRSQGPGGQGPYGPGAGQQGPGSQGPGSGGQQGPGGQGPYGP |
| | SAAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPGG |
| | QGPYGPSAAAAAAAA |

In some embodiments, the structure of molded bodies formed from the described recombinant spider silk polypeptides form beta-sheet structures, beta-turn structures, or alpha-helix structures. In some embodiments, the secondary, tertiary and quaternary protein structures of the formed molded bodies are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. While not wishing to be bound by theory, the structural properties of the proteins within the spider silk are theorized to be related to molded body mechanical properties. Crystalline regions have been linked with strength, while the amorphous regions have been linked to the extensibility. The major ampullate (MA) silks tend to have higher strengths and less extensibility than the flagelliform silks, and likewise the MA silks have higher volume fraction of crystalline regions compared with flagelliform silks.

In some embodiments, the molecular weight of the silk protein may range from 20 kDa to 2000 kDa, or greater than 20 kDa, or greater than 10 kDa, or greater than 5 kDa, or from 5 to 400 kDa, or from 5 to 300 kDa, or from 5 to 200 kDa, or from 5 to 100 kDa, or from 5 to 50 kDa, or from 5 to 500 kDa, or from 5 to 1000 kDa, or from 5 to 2000 kDa, or from 10 to 400 kDa, or from 10 to 300 kDa, or from 10 to 200 kDa, or from 10 to 100 kDa, or from 10 to 50 kDa, or from 10 to 500 kDa, or from 10 to 1000 kDa, or from 10 to 2000 kDa, or from 20 to 400 kDa, or from 20 to 300 kDa, or from 20 to 200 kDa, or from 40 to 300 kDa, or from 40 to 500 kDa, or from 20 to 100 kDa, or from 20 to 50 kDa, or from 20 to 500 kDa, or from 20 to 1000 kDa, or from 20 to 2000 kDa.

### Characterization of Recombinant Spider Silk Polypeptide Powder Impurities and Degradation

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat—Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of recombinant silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include, but are not limited to, water and polyalcohols (polyols) such as glycerol, triglycerol, hexaglycerol, and decaglycerol. Other suitable plasticizers include, but are not limited to, Dimethyl Isosorbite; biasamide of dimethylaminopropyl amine and adiptic acid; 2,2,2-trifluoro ethanol; amide of dimethylaminopropyl amine and caprylic/capric acid; DEA acetamide and any combination thereof. Other suitable plasticizers are discussed in Ullsten et. al, Chapter 5: Plasticizers for Protein Based Materials Viscoeleastic and Viscoplastic Materials (2016) (available at https://www.intechopen.com/books/viscoelastic-and-viscoplastic-materials/plasticizers-for-protein-based-materials) and Vierra et al., Natural-based plasticizers and polymer films: A review, European Polymer Journal 47(3):254-63 (2011), the entirely of these are herein incorporated by reference.

As hydrophilic portions of silk polypeptides can bind ambient water present in the air as humidity, water will almost always be present, the bound ambient water may plasticize silk polypeptides. In some embodiments, a suitable plasticizer may be glycerol, present either alone or in combination with water or other plasticizers. Other suitable plasticizers are discussed above.

In addition, in instances where recombinant spider silk polypeptides are produced by fermentation and recovered as recombinant spider silk polypeptide powder from the same, there may be impurities present in the recombinant spider silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant spider silk polypeptide powder or composition. Size Exclusion Chromatography separates molecules based on their relative size and can be used to analyze the relative amounts of recombinant spider silk polypeptide in its full-length polymeric and monomeric forms as well as the amount of high, low and intermediate molecular weight impurities in the recombinant spider silk polypeptide powder. Similarly, Rapid High Performance Liquid Chromatography may be used to measure various compounds present in a solution such as monomeric forms of the recombinant spider silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

Depending on the embodiment, the recombinant spider silk polypeptide may have a purity calculated based on the amount of the recombinant spider silk polypeptide in is monomeric form by weight relative to the other components of the recombinant spider silk polypeptide powder. In various instances, the purity can range from 50% by weight to 90% by weight, depending on the type of recombinant spider silk polypeptide and the techniques used to recover, separate and post-process the recombinant spider silk polypeptide powder.

Both Size Exclusion Chromatography and Reverse Phase High Performance Liquid Chromatography are useful in measuring full-length recombinant spider silk polypeptide, which makes them useful techniques for determining whether processing steps have degraded the recombinant spider silk polypeptide by comparing the amount of full-length spider silk polypeptide in a composition before and after processing. In various embodiments of the present invention, the amount of full-length recombinant spider silk polypeptide present in a composition before and after processing may be subject to minimal degradation. The amount of degradation may be in the range 0.001% by weight to 10% by weight, or 0.01% by weight to 6% by weight, e.g. less than 10% or 8% or 6% by weight, or less than 5% by weight, less than 3% by weight or less than 1% by weight.

### Recombinant Silk Solid and Film Compositions and Methods of Making

Depending on the embodiment, suitable concentrations of recombinant spider silk polypeptide powder by weight in the recombinant spider silk composition ranges from: 1 to 90% by weight, 3 to 80% by weight, 5 to 70% by weight, 10 to 60% by weight, 15 to 50% by weight, 18 to 45% by weight, or 20 to 41% by weight.

In some embodiments, suitable concentrations of plasticizer by weight in the recombinant spider silk composition ranges from: 1 to 60% by weight, 10 to 60% by weight, 10 to 50% by weight, 10 to 40% by weight, 15 to 40% by weight, 10 to 30% by weight, or 15 to 30% by weight. In some embodiments, the plasticizer is glycerol. In some embodiments, the plasticizer is triethanolamine, trimethylene glycol, or propylene glycol

In the instance where water is used as a plasticizer, a suitable concentration of water by weight in the recombinant spider silk composition ranges from: 5 to 80% by weight, 15 to 70% by weight, 20 to 60% by weight, 25 to 50% by weight, 19 to 43% by weight, or 19 to 27% by weight. Where water is used in combination with another plasticizer, it may be present in the range of 5 to 50% by weight, 15 to 43% by weight or 19 to 27% by weight.

After formation of the molded body, the crystallinity of the recombinant proteins in the molded body can increase, thereby strengthening the molded body. In some embodiments, the crystallinity index of the molded body as measured by X-ray crystallography is from 2% to 90%. In some other embodiments, the crystallinity index of the molded body as measured by X-ray crystallography is at least 3%, at least 4%, at least 5%, at least 6%, or at least 7%.

In some embodiments, various agents may be added to the recombinant spider silk composition to alter the characteristics of the molded body, such as hardness, flexural modulus, and flexural strength. These include polyethylene glycol (PEG), Tween (polysorbate), sodium dodecyl sulfate, polyethylene, or any combination thereof. Other suitable agents are well known in the art.

In some embodiments, a second polymer may be added to create a polymer blend or bi-constituent fiber with the recombinant spider silk composition. In these instances, it may be useful to include a second polymer that has a melting temperature that makes it suitable for melting, in tandem with the recombinant spider silk composition itself, without degrading the amorphous regions of the recombinant spider silk polypeptide. In various embodiments, polymers suitable for blending with recombinant spider silk polypeptides will have a melting temperature (Tm) of less than 200° C., 180° C., 160° C., 140° C., 120° C. or 100° C. Often, the recombinant spider silk polypeptide will have a melting temperature of more than 20° C., or 25° C. or 50° C. A non-limiting list of exemplary polymers and the melting temperatures is included in Table 3 below.

TABLE 3
Polymers

| Polymer | Tm ° C. |
|---|---|
| LLDPE, Linear Low Density Polyethylene | 120-130 |
| LDPE, Low Density Polyethylene | 105-120 |
| MDPE, Medium Density Polyethylene | 120-180 |
| HDPE, High Density Polyethylene | 130+ |
| PP, Polypropylene | 130+ |
| PLA PolyLactic Acid | 125-175 |
| EVA Ethyl Vinyl Acetate | 70-85 |
| PBAT Poly(butylene adipate-co-terephthalate) | 110-120 |
| PBSA Polybutylene Succinate Adipate | 116 |
| PBS Polybutylene Succinate | 84-115 |
| DuPont ™ Ionomers (e.g. Surlyn ® ionomers) | 80-100 |
| EPE, Expanded Polyethylene | 126 |
| PC Polycarbonate | 155 |
| PCL Polycaprolactone | 60 |

In some embodiments, water may be evaporated during cooling or post-molding conditioning. In some embodiments, water loss after molding may range from 1 to 50% by weight, 3 to 40% weight, 5 to 30% weight, 7 to 20% weight, 8 to 18% weight, or 10-15% based on the total water amount. Often loss will be less than 15%, in some cases less than 10%, for instance 1 to 10% by weight. Evaporation may be intentional or as a result of the treatment applied. The degree of evaporation can be easily controlled, for instance by selection of operating temperatures, flow rates and pressures applied, as would be understood in the art.

In some embodiments, suitable plasticizers may include polyols (e.g., glycerol), water, lactic acid, methyl hydroperoxide, ascorbic acid, 1,4-dihydroxybenzene (1,4 benzenediol) benzene-1,4-diol, phosphoric acid, ethylene glycol, propylene glycol, triethanolamine, acid acetate, propane-1,3-diol or any combination thereof.

In various embodiments, the amount of plasticizer can vary according to the purity and relative composition of the recombinant spider silk polypeptide powder. For example, a higher purity powder may have less impurities such as a low molecular weight compounds that may act as plasticizers and therefore require the addition of a higher percentage by weight of plasticizer.

In specific embodiments, various ratios (by weight) of the plasticizer (e.g. a combination of glycerol and water) to the recombinant spider silk polypeptide powder may range from 0.5 or 0.75 to 350% by weight plasticizer: recombinant spider silk polypeptide powder, 1 or 5 to 300% by weight plasticizer: recombinant spider silk polypeptide powder, 10 to 300% by weight plasticizer: recombinant spider silk polypeptide powder, 30 to 250% by weight plasticizer: recombinant spider silk polypeptide powder, 50 to 220% by weight plasticizer: recombinant spider silk protein, 70 to 200% by weight plasticizer: recombinant spider silk polypeptide powder, or 90 to 180% by weight plasticizer: recombinant spider silk polypeptide powder. As used herein, reference to 0.5 to 350% by weight plasticizer:recombinant spider silk polypeptide powder corresponds to a ratio of 0.5:1 to 350:1.

Without intending to be limited by theory, in various embodiments of the present invention, inducing the recombinant spider silk composition to transition into a flowable state may be used as a pre-processing step in any formulation in circumstances where it is beneficial to include the recombinant spider silk polypeptide in its monomeric form. More specifically, inducing the recombinant spider silk melt composition may be used in applications where it is desirable to prevent the aggregation of the monomeric recombinant spider silk polypeptide into its crystalline polymeric form or to control the transition of the recombinant spider silk polypeptide into its crystalline polymeric form at a later stage in processing. In one specific embodiment, the recombinant spider silk melt composition may be used to prevent aggregation of the recombinant spider silk polypeptide prior to blending the recombinant spider silk polypeptide with a second polymer. In another specific embodiment, the recombinant spider silk melt composition may be used to create a base for a cosmetic or skincare product where the recombinant spider silk polypeptide is present in the base in its monomeric form. In this embodiment, having the recombinant spider silk polypeptide in its monomeric form in a base allows for the controlled aggregation of the monomer into its crystalline polymeric form upon contact with skin or through various other chemical reactions.

The cosmetic or skincare product may be applied directly to the skin or hair. In some embodiments, the molded body has a low melting temperature. In various embodiments, the molded body has a melting temperature that is less than body temperature (around 34-36° C.) and melts upon contract with skin.

The cosmetic or skincare products discussed above may contain various humectants, emollients, occlusive agents, active agents and cosmetic adjuvants, depending on the embodiment and the desire efficacy of the product.

The term “humectant” as used herein refers to a hygroscopic substance that forms a bond with water molecules. Suitable humectants include but are not limited to glycerol, propylene glycol, polyethylene glycol, pentalyene glycol, tremella extract, sorbitol, dicyanamide, sodium lactate, hyaluronic acid, aloe vera extract, alpha-hydroxy acid and pyrrolidonecarboxylate (NaPCA). The term “emollient” as used herein refers to a compound that provide skin a soft or supple appearance by filling in cracks in the skin surface. Suitable emollients include but are not limited to shea butter, cocao butter, squalene, squalane, octyl octanoate, sesame oil, grape seed oil, natural oils containing oleic acid (e.g. sweet almond oil, argan oil, olive oil, avocado oil), natural oils containing gamma linoleic acid (e.g. evening primrose oil, borage oil), natural oils containing linoleic acid (e.g. safflower oil, sunflower oil), or any combination thereof. The term “occlusive agent” refers to a compound that forms a barrier on the skin surface to retain moisture. In some instances, emollients or humectants may be occlusive agents. Other suitable occlusive agents may include but are not limited to beeswax, canuba wax, ceramides, vegetable waxes, lecithin, allantoin. Without being limited to theory, the film-forming capabilities of the recombinant spider silk compositions presented herein make an occlusive agent that forms a moisture retaining barrier because the recombinant spider silk polypeptides act attract water molecules and also act as humectants.

The term “active agent” refers to any compound that has a known beneficial effect in skincare formulation or sunscreen. Various active agents may include but are not limited to acetic acid (i.e. vitamin C), alpha hydroxyl acids, beta hydroxyl acids, zinc oxide, titanium dioxide, retinol, niacinamide, other recombinant proteins (either as full length sequences or hydrolyzed into subsequences or “peptides”), copper peptides, curcuminoids, glycolic acid, hydroquinone, kojic acid, 1-ascorbic acid, alpha lipoic acid, azelaic acid, lactic acid, ferulic acid, mandelic acid, dimethylaminoethanol (DMAE), resveratrol, natural extracts containing antioxidants (e.g. green tea extract, pine tree extract), caffeine, alpha arbutin, coenzyme Q-10, and salicylic acid. The term “cosmetic adjuvant” refers to various other agents used to create a cosmetic product with commercially desirable properties including without limitation surfactants, emulsifiers, preserving agents and thickeners.

In various embodiments, the temperature to which the recombinant spider silk composition is heated to during molding will be minimized in order to minimize or entirely prevent degradation of the recombinant spider silk polypeptide. In specific embodiments, the recombinant spider silk melt will be heated to a temperature of less than 120° C., less than 100° C., less than 80° C., less than 60° C., less than 40° C., or less than 20° C. Often the melt will be at a temperature in the range 10° C. to 120° C., 10° C. to 100° C., 15° C. to 80° C., 15° C. to 60° C., 18° C. to 40° C. or 18 to 22° C. during molding.

In some embodiments of the present invention, the recombinant spider silk solid or film will be substantially homogeneous meaning that the material, as inspected by light microscopy, has a low amount or does not have any inclusions or precipitates. In some embodiments, light microscopy may be used to measure birefringence which can be used as a proxy for alignment of the recombinant spider silk into a three-dimensional lattice. Birefringence is the optical property of a material having a refractive index that depends on the polarization and propagation of light. Specifically, a high degree of axial order as measured by birefringence can be linked to high tensile strength. In some embodiments, recombinant spider silk solids and films will have minimal birefringence.

The amount of degradation of the recombinant spider silk polypeptide may be measured using various techniques. As discussed above, the amount of degradation of the recombinant spider silk polypeptide may be measured using Size Exclusion Chromatography to measure the amount of full-length recombinant spider silk polypeptide present. In various embodiments, the composition is degraded in an amount of less than 6.0 weight % after it is formed into a molded body. In another embodiment, the composition is degraded in an amount of less than 4.0 weight % after molding, less than 3.0 weight %, less than 2.0 weight %, or less than 1.0 weight % (such that the amount of degradation may be in the range 0.001% by weight to 10%, 8%, 6%, 4%, 3%, 2% or 1% by weight, or 0.01% by weight to 6%, 4%, 3%, 2% or 1% by weight). In another embodiment, the recombinant spider silk protein in the melt composition is substantially non-degraded.

In some embodiments, the molded body is cross-linked. For example, in some embodiments, during or after forming a molded body, the molded body is soaked in ammonium persulfate to facilitate cross-linking between proteins in the molded body. In some embodiments, said cross-linking is enzymatic cross-linking. In some embodiments, said cross-linking is photochemical cross-linking.

In some embodiments, provided herein are cross-linked recombinant silk molded bodies with desirable mechanical properties and methods of producing them. The cross-linked molded body compositions provided herein can be cross-linked to achieve desired mechanical properties, such as flexibility, hardness, or strength that are preferred in certain applications. In some embodiments, provided herein are methods of cross-linking recombinant silk molded body compositions to form a cross-linked recombinant silk solid. In some embodiments, the cross-linking reaction comprises exposure of the molded body to a persulfate, such as ammonium persulfate. Heat can be applied to initiate a cross-linking reaction catalyzed by persulfate. This type of cross-linking reaction does not leave any photoactive or enzymatic compounds in the composition. Furthermore, this cross-linking reaction does not require photoactivation, so large batches can be produced efficiently without the requirement for light to reach all parts of the cross-linking solution. In some embodiments, cross-linking occurs in vessels or molds such that the recombinant silk molded bodies obtained have specific shapes or forms.

In some embodiments, the molded body is formed via 3D printing. Thus, in some embodiments, the molded body is formed by depositing or forming thin layers of a composition comprising recombinant silk and plasticizer in a flowable state in succession so as to build up a desired 3-D structure. Each layer is formed as if it were one layer of printing, e.g. by moving some kind of printing head over a workpiece and activating elements of the printing head to create the “printing”. polymerisable liquid material. Thus, in some embodiments, a molded body is formed layer by layer. Each layer comprises a dispersed composition comprising the recombinant silk and plasicizer in a flowable state, and the dispersed composition is cross-linked or hardened in a pattern which is the same as a cross-section through the object to be formed. After one layer is completed the level of distributed composition is raised over a small distance and the process repeated. Each polymerised layer should be sufficiently form stable to support the next layer.

In another embodiment, the composition comprising recombinant silk and plasticizer is distributed onto a substrate and coalesced, in accordance with the shape of the cross-section of the object to be formed. In yet another embodiment, the composition comprising recombinant silk and plasticizer is deposited in the form of drops which are deposited in a pattern according to the relevant cross-section of the object to be formed. Still another method involves dispensing drops of the composition at an elevated temperature which then solidify on contact with the cooler work piece.

### Re-Forming of Recombinant Silk Solids and Films

In some embodiments of the present invention, the process for preparing the recombinant spider silk molded body may additionally comprise re-processing a molded body comprising the recombinant spider silk (e.g. a solid, film, or other molded article formed from recombinant spider silk).

Without intending to be limited by theory, subjecting the recombinant spider silk polypeptide to heat and pressure in the presence of a plasticizer such as glycerol converts the recombinant spider silk polypeptide into an “open-form recombinant spider silk polypeptide” in which the uncrystallized and amorphous recombinant spider silk polypeptide segments unfold and form interactions with the plasticizer. Due to the interactions with plasticizer, this “open-form recombinant spider silk polypeptide” enables molding and forming a solid. Specifically, the open form recombinant spider silk polypeptide is prevented from forming intermolecular interactions to form an irreversible three-dimensional lattice.

Because there is minimal degradation (if any) of the recombinant spider silk polypeptide during the molding process, in some embodiments, the recombinant spider silk molded body is reprocessed by transforming the molded body back into a flowable recombinant spider silk composition, which is then re-molded. In various embodiments, the recombinant spider silk molded body may be re-molded at least 20 times, at least 10 times, or at least 5 times. In these embodiments, the degradation seen over multiple re-molding steps may be as low as 10%. The option of re-molding without degradation allows for the production of substantially homogeneous compositions, and also for the repurposing or redesign of products formed from the composition. For instance, molded products which are of insufficient quality, may be remolded. End of life product recycling is also a possibility.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B(1992).

### Example 1: Formation of Recombinant Silk Protein Solids

Beta sheets play an important role in structural integrity of silk materials. They make up the crystalline segments of the silk. Typically, when the beta sheets are formed, strong chaotropic solvents are required to disrupt the beta sheets. The melting temperature of beta sheets is above its degradation point. However, the glass transition temperature is lower than the degradation temperature and can be further reduced with the use of plasticizers.

To make solids, adequate entanglement is required. The melting temperature of the beta-sheets is too high, but since the majority of the protein is amorphous, it is possible to provide chain mobility to the amorphous chains to allow adequate entanglement. Application of heat and plasticizers can reduce the thermal glass transition temperature. The three components necessary for obtaining 18B solids were heat, pressure and plasticizer.

A recombinant spider silk of the 18B polypeptide sequence (SEQ ID NO: 1) was produced through various lots of large-scale fermentation, recovered and dried in powders (“18B powder”). Details of production of 18B recombinant silk powder are found in PCT Publication No. WO2015/042164, “Methods and Compositions for Synthesizing Improved Silk Fibers,” incorporated herein by reference in its entirety. The recombinant silk powder was mixed using a household spice grinder. Ratios of water and plasticizer were added to 18B powder to generate recombinant spider silk compositions with different ratios of protein powder to plasticizer. The resulting composition was 10-50% by weight triethanolamine (TEOA), trimethlylene glycol, or propylene glycol. The mixture was then pressed at 130° C. Pressure in the range of 1500 to 15000 psi were used to press samples in a mold.

At 30% by weight TEOA, during pressing some of the TEOA plasticizer squeezed out of the mold as shown in FIG. 1. This suggests that the TEOA amount can be lowered if the TEOA can be evenly distributed throughout the powder. Pressure was used to compact the powder particles.

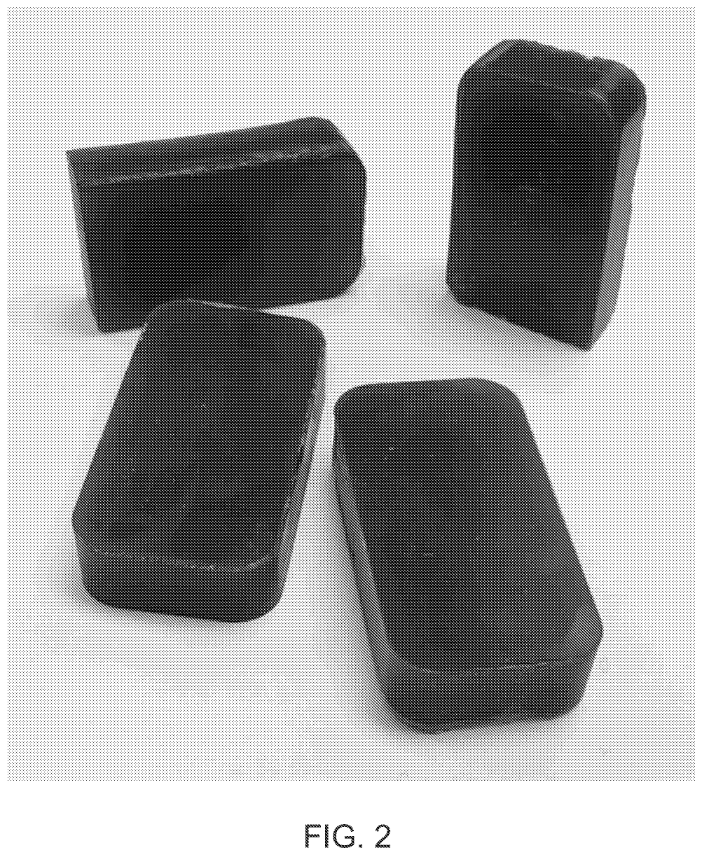

The hardness of the solids was measured with a durometer. The durometer has an indenter that penetrates into the material. The larger the penetration the softer the material and the lower the measured hardness value. There are multiple types of durometers which are intended for various ranges for hardness. Type A durometers are for soft plastics and if the value exceeds 90, type D durometer should be used. The difference between them is the indenter geometry and the applied force. As durometer D is intended for harder plastics it has a sharper indenter and higher indentation force. Solids pressed with TEOA, propylene glycol, trimethylene glycol (1,3 propanediol) all had a hardness of 100 when measured with Type A, indicating that their hardness exceeds hardness measurable by type A durometer. TEOA processed solid had a hardness of 76 HD as measured by a type D durometer. Trimethylene glycol processed solids had a hardness of 71 HD as measured by a type D durometer (FIG. 2). For comparison, a high-density polyethylene (HDPE) hard hat has a similar hardness. Propylene glycol solids had the lowest hardness, starting at 55 HD and dropping to 30 within 10 seconds as measured by a type D durometer. The solids could be machined, cut and drilled into desired shapes as their rigidity prevented the solids from deforming under the tool force (FIG. 2).

### Example 2: Degradation of Recombinant Silk in Silk Solids

SEC results for pressed films and solids showed a similar low and intermediate molecular weight between the solid sample, film samples (see Example 5) and control 18B powder. This suggested that degradation caused by pressing was minimal or nonexistent. Table 4. SEC data of pressed solids and films along with control powder. Average results and standard deviation of N=2. HMWI=High Molecular Weight Impurities; IMWI=Intermediate Molecular Weight Impurities; LMWI=Low Molecular Weight Impurities. All samples were from the same 18B powder lot. The solid was pressed with 30 wt % (% by weight) TEOA, and the films were pressed with 40 wt % glycerol.

TABLE 4
Composition of recombinant silk solids and precursors

| | Sum HMWI, | | 18B | 18B | | |
|---|---|---|---|---|---|---|
| | 18B agg and | HMWI | aggregate | monomers | IMWI | LMWI |
| Sample* | mon (%) | (%) | (%) | (%) | (%) | (%) |
| Solid | 57.32 | 7.2 ± 0.78 | 11.79 ± 0.78 | 38.33 ± 2.15 | 34.6 ± 0.55 | 8.08 ± 4.26 |
| Film 1 | 57.87 | 4.55 ± 0.01 | 9.66 ± 0.89 | 43.66 ± 5.76 | 32.63 ± 1.57 | 9.51 ± 5.07 |
| Film 2 | 56.06 | 5.29 ± 0.2 | 10.17 ± 0.53 | 40.6 ± 1.78 | 35.52 ± 0.89 | 8.42 ± 1.22 |
| 18B | 59.56 | 3.65 ± 0.13 | 8.53 ± 0.28 | 47.38 ± 5.41 | 34.36 ± 3.21 | 6.08 ± 2.61 |
| Powder | | | | | | |

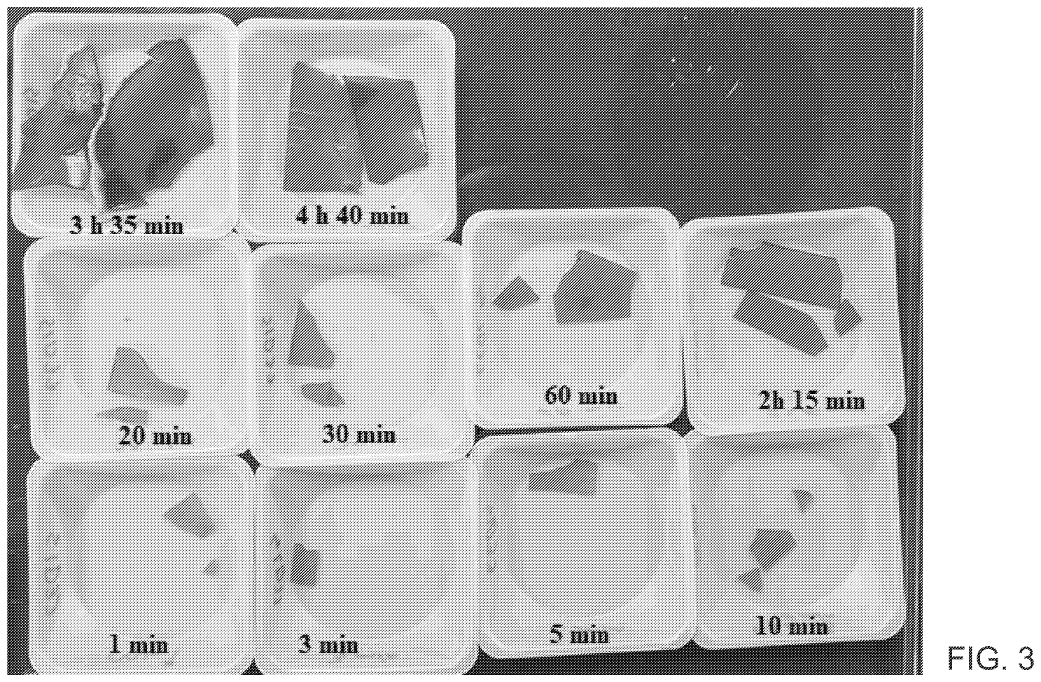

Protein degradation data is summarized in Table 5. Here, the sample was heated at 130° C. and pressed for increasing times. At each time point, the solid was sampled and placed back in the mold, where heat and pressure was applied. Based on the HMWI, 18B aggregate and 18B monomer, and the IMWI and LMWI values between samples, there was not significant degradation up to 10 minutes. From 20 minutes onward, the 18B monomer content dropped while the intermediate (IMWI) and low (LMWI) molecular weight components increased, suggesting degradation beyond 20 minutes. As the solid was pressed for alonger time, it also became darker (FIG. 3).

TABLE 5
SEC data of control powder (SLD33-P), powder plasticized with solvent
(SLD33-PH), and pressed solids (SLD33) for increasing press times. All
samples were from the same 18B powder lot. The solid was pressed with
15% by weight 1,3 propanediol.

| | Sum | | | | | |
|---|---|---|---|---|---|---|
| | HMWI, | | | | | |
| | 18B agg | | 18B | 18B | | |
| | and mon | HMWI | aggregate | monomers | IMWI | LMWI |
| Sample | (%) | (%) | (%) | (%) | (%) | (%) |
| SLD33-P | 66.04 | 2.47 | 8.15 | 55.42 | 24.29 | 9.68 |
| SLD33-PH | 69.21 | 2.55 | 7.20 | 59.46 | 24.05 | 6.74 |
| SLD33-1 min | 62.92 | 3.11 | 7.86 | 51.94 | 25.64 | 11.45 |
| SLD33-3 min | 69.02 | 4.00 | 9.43 | 55.59 | 23.58 | 7.40 |
| SLD33-5 min | 64.57 | 3.92 | 10.21 | 50.43 | 25.06 | 10.37 |
| SLD33-10 min | 67.83 | 6.13 | 11.72 | 49.98 | 24.68 | 7.48 |
| SLD33-20 min | 62.51 | 6.36 | 13.83 | 42.32 | 26.47 | 11.02 |
| SLD33-30 min | 61.43 | 8.03 | 14.88 | 38.52 | 30.06 | 8.51 |
| SLD33-60 min | 52.10 | 5.44 | 19.82 | 26.83 | 35.09 | 12.82 |
| SLD33-2 hr | 44.95 | 7.03 | 19.46 | 18.46 | 41.23 | 13.82 |
| 15 m | | | | | | |
| SLD33-3 hr | 33.53 | 4.17 | 17.89 | 11.47 | 47.05 | 19.42 |
| 35 min | | | | | | |
| SLD33-4 hr | 34.03 | 6.57 | 17.86 | 9.60 | 44.75 | 21.22 |
| 40 min | | | | | | |

### Example 3: Flexural Characterization of 18B Solids

18B protein powder has shown promising capabilities as a stable protein powder with desirable solid characteristics when sintered via compression molding as described herein (e.g., in Example 1). Trimethylene glycol (TMG or 1,3-propanediol) was identified as a suitable plasticizer to assist in molding. For the purpose of optimizing the molding process, further characterization of the mechanical properties of 18B-TMG solids was required. Batches of 18B with 15% by weight TMG solid powder were created and subjected to 3-point bend testing per ASTM D790.

As described below, mechanical properties of 18B solids across a range of processing parameters were provided including molding hold time, cooling rate, post-mold conditioning, and average pressing load. Processing parameters that were beneficial or detrimental to the mechanical properties of the final solid product were also discovered, thereby improving processing efficiency and capability.

### Materials and Methods

For testing flexural characteristics of the recombinant silk solids, ASTM D790 standard recommends a span-to-depth (thickness) ratio as close to 16:1 as possible, while the Zwick recommends keeping the span-to-depth ratio between 15:1 and 17:1. For this experiment, the span of the apparatus was fixed at 38.1 mm, such that the final specimen depth was between 2.25 mm and 2.54 mm.

Using a 25.4 mm×50.8 mm (1″×2″) compression mold resulted in 0.66 mm of thickness per final weight in grams in the solid. Based on the observation of about a 10% reduction in weight during molding, a pre-mold weight per specimen of 3.8 g to 4.0 g was used to achieve the final specimen depth.

An 18B/TMG mixture was prepared using 255.16 g 18B powder and 45.347 g TMG, which was mixed five times using a spice grinder, yielding a 300.5 g total master batch of 15.1% by weight TMG/84.9% by weight 18B. These were separated into specimens of 4.0 g each for molding under defined conditions and subsequent testing of flexural characteristics.

Over the 63 specimens used for testing, the average span to depth ratio was 15.72 with a standard deviation of 0.35 resulting in a coefficient of variation of 0.022. Testing configuration on Zwick ProLine was performed according to ASTM D790 testing program file. Key testing parameters were 0.1 MPa pre-load, 3 mm starting position separation, and a crosshead speed of 254 mm/min.

Recombinant silk solid preparation conditions tested were mold time, cooling rate, post-mold conditioning, and average load during molding.

Mold time is defined as time (in minutes) the mold is under compression at 130° C. Mold times of 1 minute, 2 minutes, 3 minutes, 4 minutes, 5 minutes, 6 minutes, 8 minutes, 10 minutes, and 15 minutes were tested.

For post-mold conditioning, a conditioned sample remained in the conditioning chamber for a minimum of 72 hours at 65% relative humidity (RH) after the mold time. A specimen without conditioning was stored on top of workbench under ambient lab conditions.

The average load was the load in metric tons the specimen was subjected to. Because the specimen size and mold size were constant, each specimen in a sample group was subjected to near-equivalent pressure during molding. Average loads of 1 metric ton, 2 metric tons, 3 metric tons, 4 metric tons, and 5 metric tons were tested.

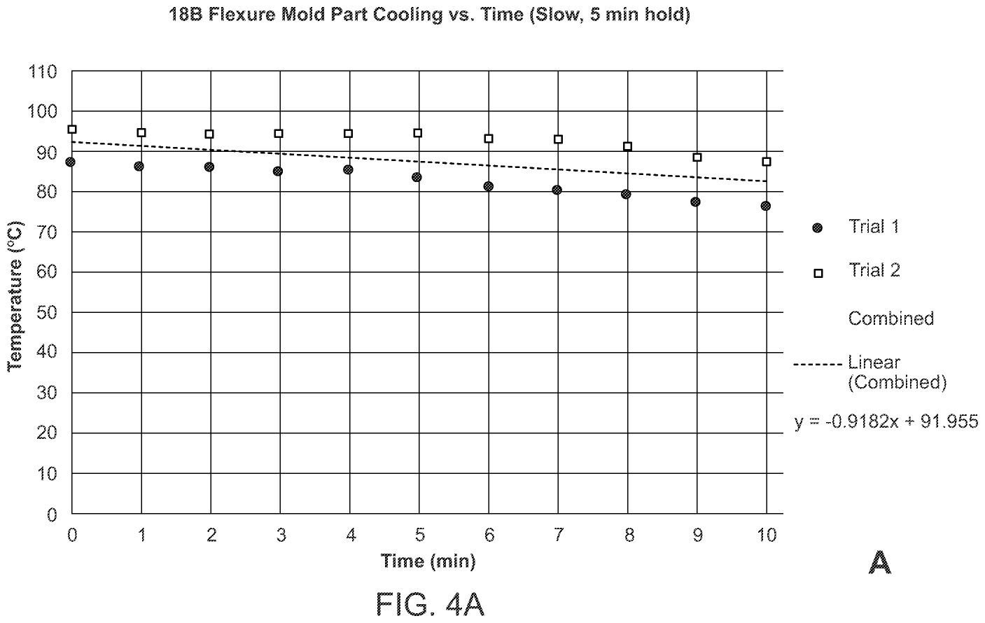

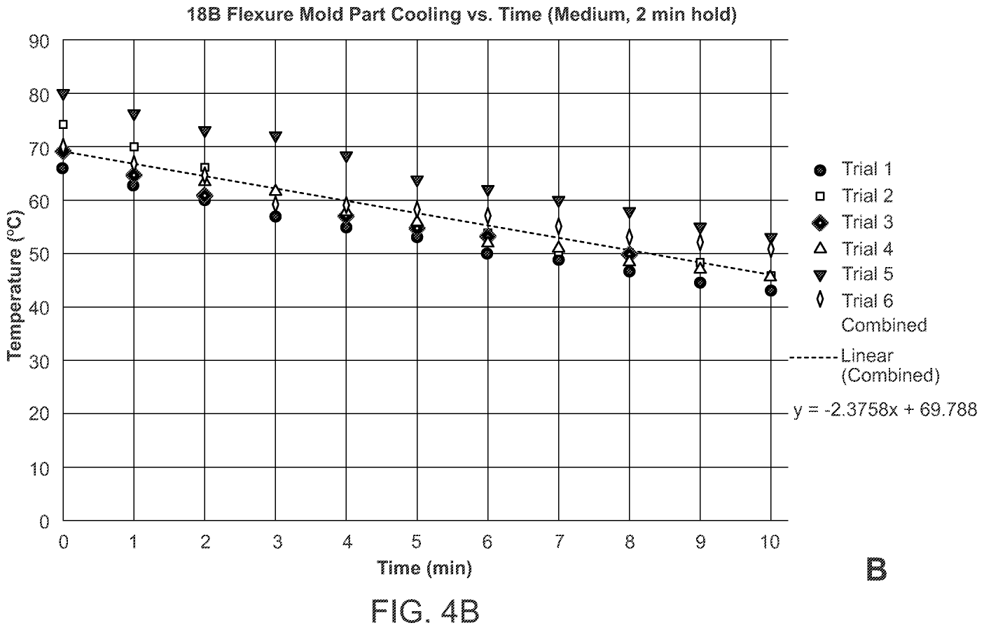

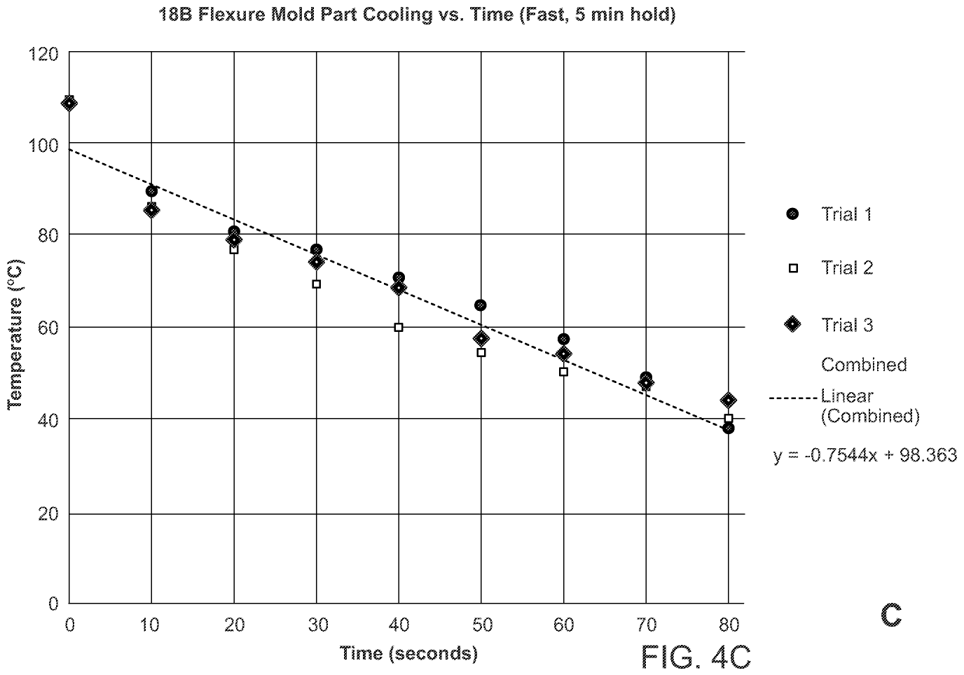

Finally, the cooling rate levels were defined as either slow, medium, or fast. Each level was quantified using an IR thermometer recording solid surface temperature either at 1-minute intervals (slow, medium) or 10-second intervals (fast) beginning when the mold was opened to remove a solid specimen. The results from the curves shown below yielded cooling rates of 0.92° C./min, 2.7° C./min, and 45.2° C./min for slow, medium, and fast, respectively. Though in FIGS. 4A-4C, the samples with medium cooling rates were at a different hold time compared to that with slow and fast cooling rates, the rate of cooling did not substantially differ with hold time. Cooling rates of slow, medium, and fast as defined above were tested.

Table 6 below shows conditions used for preparation of each sample ID. Each sample ID was performed in triplicate for a total of 63 18B solid samples prepared.

TABLE 6
Sample ID Preparation Conditions

| Sample | Mold Time | Cooling | | Average Load |
|---|---|---|---|---|
| ID# | (min) | Rate | Conditioning | (metric tons) |
| 1 | 1 | Medium | 65% RH | 2 |
| 2 | 1 | Medium | None | 2 |
| 3 | 2 | Medium | 65% RH | 2 |
| 4 | 2 | Medium | None | 2 |
| 5 | 3 | Medium | 65% RH | 2 |
| 6 | 3 | Medium | None | 2 |
| 7 | 4 | Medium | 65% RH | 2 |
| 8 | 4 | Medium | None | 2 |
| 9 | 5 | Medium | 65% RH | 2 |
| 10 | 5 | Slow | None | 2 |
| 11 | 5 | Medium | None | 2 |
| 12 | 5 | Fast | None | 2 |
| 13 | 5 | Medium | None | 1 |
| 14 | 5 | Medium | None | 2 |
| 15 | 5 | Medium | None | 3 |
| 16 | 5 | Medium | None | 4 |
| 17 | 5 | Medium | None | 5 |
| 18 | 6 | Medium | None | 2 |
| 19 | 8 | Medium | None | 2 |
| 20 | 10 | Medium | None | 2 |
| 21 | 15 | Medium | None | 2 |

Post-Mold Conditioning

18B Solid samples were molded as described above using 4.0 g samples and molded at 130° C. under an average load of 2 metric tons. Molded samples were cooled at a medium cooling rate and exposed or not exposed to conditioning at 65% relative humidity (RH) for a minimum of 72 hours after molding. Samples were molded for 1, 2, 3, 4, or 5 minutes. The conditions for assessing the effect of post-mold conditioning were based on samples 1-9 and 11 provided in Table 6.

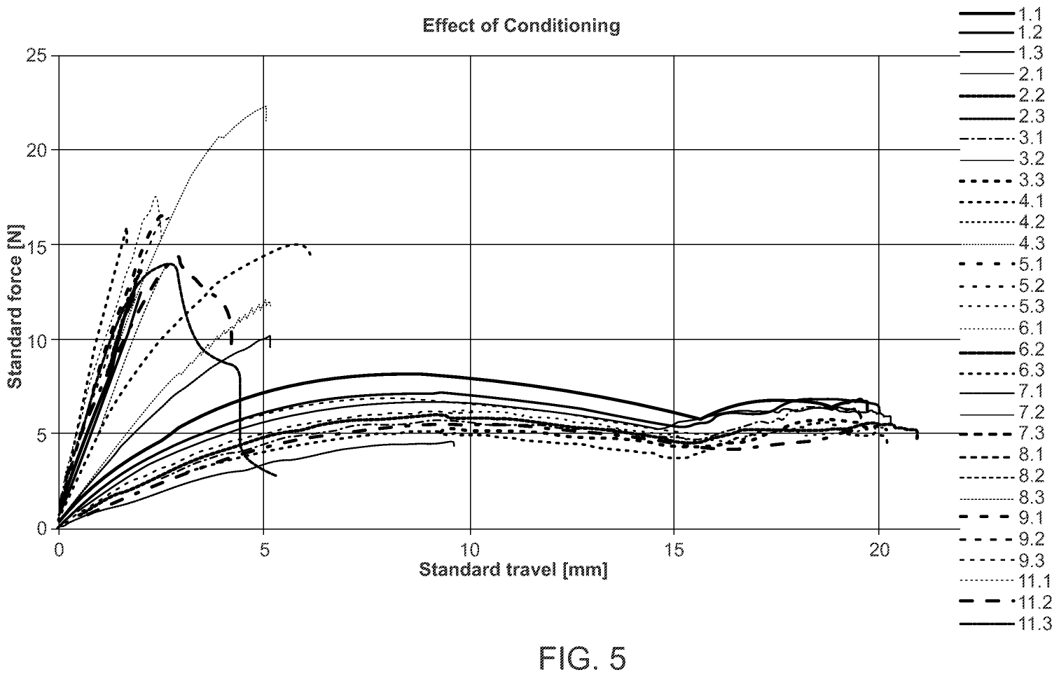

FIG. 5 shows stress-strain curves generated from unconditioned 18B solid samples vs. conditioned 18B solid samples. The stress-strain curves were used to determine the mechanical properties of 18B solids, including elongation at break. Sample ID 1, 3, 5, 7, and 9 were conditioned and Sample ID 2, 4, 6, 8, and 11 were not conditioned, as shown in Table 6 and Table 7.

Flexural data for conditioned vs. unconditioned 18B solid samples are shown in Table 7 below. Average values of flexural modulus (MPa), maximum flexural strength (MPa), and elongation at break (%) for each of the conditioned vs. unconditioned samples measured in triplicate (along with measured standard deviation (SD)) are provided. Note that an elongation of 20% indicates no breakage of the solid as 20% was the maximum testable elongation.

TABLE 7
Conditioned vs. Unconditioned 18B Solid Samples (Flexural Data)

| | | | | | Average | | | |
|---|---|---|---|---|---|---|---|---|
| | | | Average | SD | Max. | SD Max. | Average | SD |
| | Mold | | Flexural | Flexural | Flexural | Flexural | Elongation | Elongation |
| Sample | Time | | Modulus | Modulus | Strength | Strength | at Break | at Break |
| ID# | (min) | Conditioning | (MPa) | (MPa) | (MPa) | (MPa) | (%) | (%) |
| 1 | 1 | 65% RH | 36.95 | 9.95 | 1.83 | 0.18 | 20.00 | 0.00 |
| 2 | 1 | None | 193.44 | 77.14 | 4.51 | 0.32 | 2.97 | 1.86 |
| 3 | 2 | 65% RH | 77.31 | 12.04 | 2.73 | 0.26 | 20.00 | 0.00 |
| 4 | 2 | None | 220.12 | 155.59 | 4.93 | 0.99 | 3.19 | 1.81 |
| 5 | 3 | 65% RH | 55.25 | 14.53 | 2.42 | 0.28 | 20.00 | 0.00 |
| 6 | 3 | None | 252.80 | 18.00 | 5.39 | 2.51 | 2.79 | 2.13 |
| 7 | 4 | 65% RH | 41.08 | 9.91 | 2.12 | 0.31 | 20.00 | 0.00 |
| 8 | 4 | None | 255.06 | 65.23 | 5.61 | 0.34 | 4.70 | 1.84 |
| 9 | 5 | 65% RH | 49.65 | 9.86 | 2.31 | 0.27 | 20.00 | 0.00 |
| 11 | 5 | None | 309.54 | 52.43 | 6.11 | 0.70 | 3.15 | 0.94 |

FIG. 6 shows the morphology of solids subjected to 1-minute hold time (L) conditioned for 72 hours in 65% RH environment and (R) unconditioned. The solids had comparable particle sizes, though the conditioned specimen had more clearly amorphous regions between particles possibly lending to increased ductility.

Macroscopically, it was evident that all 65% RH conditioned samples were more ductile under load compared to unconditioned counterparts. The two hydroxyl groups present in the TMG plasticizer contributed to increased water solubility and hygroscopicity. Less time in mold yielded solids that had a powder-like morphology and contained more particles, as shown in FIG. 6.

Based on the effects of conditioning, there was a tradeoff between stiffness and elongation for conditioned samples. Conditioning specimens created specimens that were not very strong nor stiff and would not fracture. The safety limits of the testing apparatus required the test to stop at a maximum 20% elongation, and none of the conditioned samples fractured up to that elongation. Unconditioned samples also had more variability. The effect of conditioning was pronounced for uncross-linked 18B solids, suggesting they are susceptible to exposure to water. Thus, crosslinked 18B solids will be generated to reduce the response of 18B solids to water. Mechanical characteristics of strength, stiffness, and elongation for the cross-linked 18B solids will also be maximized.

Conditioned samples did not fracture because their elongation percentage exceeded the safety measures in place by the Zwick ProLine. For that reason, conditioned sample fracture surfaces could not be assessed. Macroscopic (visually by eye) post-fracture viewing of the unconditioned sample fracture surfaces revealed that nearly all flexural fractures could be characterized as highly brittle with minor varying degrees of ductile behavior depending on the processing. Initiation was typically within 0.5 cm of the center of the specimen's width. SEM imaging of three surfaces confirmed these conclusions.

Post-Mold Cooling Rate

18B Solid samples were molded as described above using 4.0 g samples and molded for 5 minutes at 130° C. under an average load of 2 metric tons. Molded samples were cooled at a slow, medium, or fast cooling rate. The methodology for measuring cooling rate and the quantitative basis for slow, medium, and fast cooling is explained above in Materials and Methods. The conditions for assessing the effect of cooling rate are based on samples 10-12 provided in Table 6.

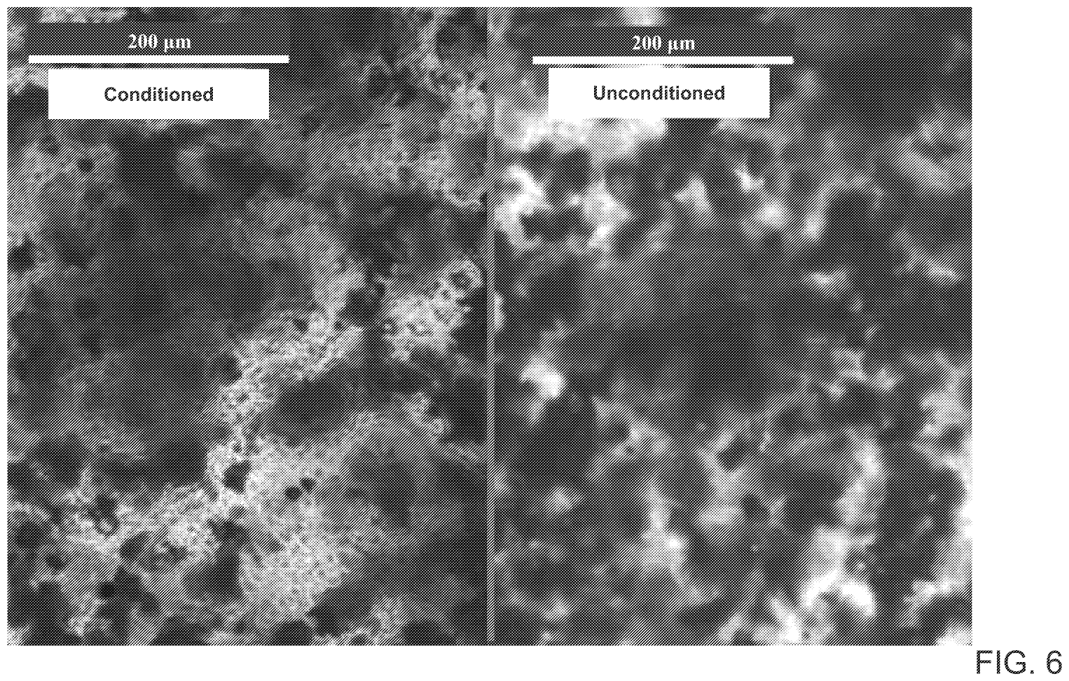

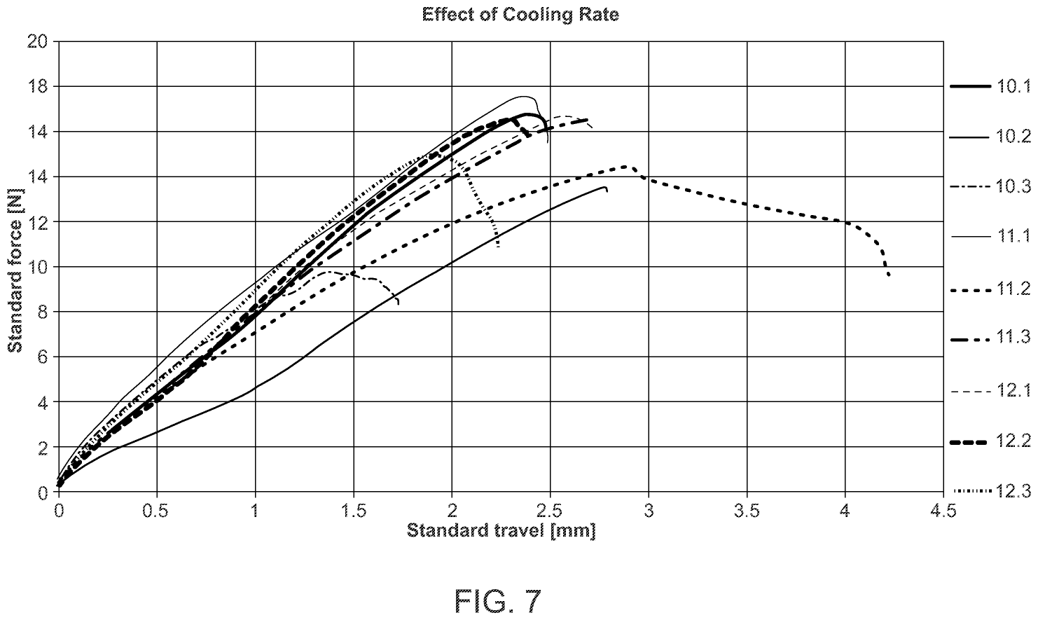

FIG. 7 shows stress strain curves generated from samples 10-12 to assess the effect of cooling rate on the mechanical properties of 18B solids. The 10, 11, and 12 series correspond to slow, medium, and fast cooling rates, respectively.

Flexural data for 18B solid samples at slow, medium, and fast cooling rates are shown in Table 8 below. Average values of flexural modulus (MPa), maximum flexural strength (MPa), and elongation at break (%) for each of the conditioned vs. unconditioned samples measured in triplicate (along with measured standard deviation (SD)) are provided.

TABLE 8
Effect of Cooling Rate on 18B Solid Samples (Flexural Data)

| | | Average | SD | Average Max. | SD Max. | Average | SD |
|---|---|---|---|---|---|---|---|
| | | Flexural | Flexural | Flexural | Flexural | Elongation | Elongation |
| Sample | | Modulus | Modulus | Strength | Strength | at Break | at Break |
| ID# | Cooling Rate | (MPa) | (MPa) | (MPa) | (MPa) | (%) | (%) |
| 10 | Slow | 262.72 | 81.58 | 5.29 | 1.30 | 2.28 | 0.53 |
| 11 | Medium | 309.54 | 52.43 | 6.11 | 0.70 | 3.15 | 0.94 |
| 12 | Fast | 292.35 | 18.82 | 6.12 | 0.37 | 2.45 | 0.27 |

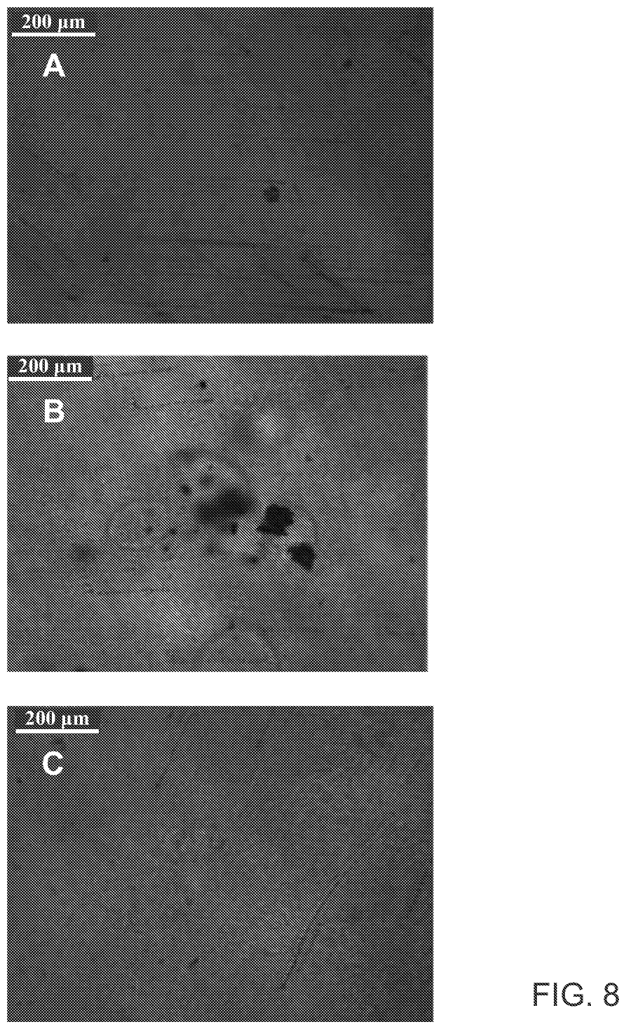

FIG. 8 shows the morphology of 18B solids exposed to (A) slow cool (B) medium cool and (C) fast cool.

In structural polymers, increased cooling rate yields stronger and stiffer samples with relatively similar elongation. Faster cooling leads to smaller crystals and less crystallinity (more amorphous regions), so one would expect less rigidity. However, the current results conflict with that assumption. On average, slow cooling had a flexural modulus and maximum strength of 262.72 MPa and 5.29 MPa, respectively. On average, medium cooling samples yielded a flexural modulus and maximum strength of 309.54 MPa and 6.11 MPa, respectively. Fast cooled samples, on average, had a flexural modulus and maximum strength of 292.35 MPa and 6.12 MPa, respectively. Variability decreased as cooling rate increased.

Mold Pressure

18B solid samples were molded as described above using 4.0 g samples and molded at 130° C. for 5 minutes, followed by cooling at a medium cooling rate. Samples were molded under an average load of 1 metric ton, 2 metric tons, 3 metric tons, 4 metric tons, or 5 metric tons. The conditions for assessing the effect of average load pressure during molding are based on samples 13-17 provided in Table 6.

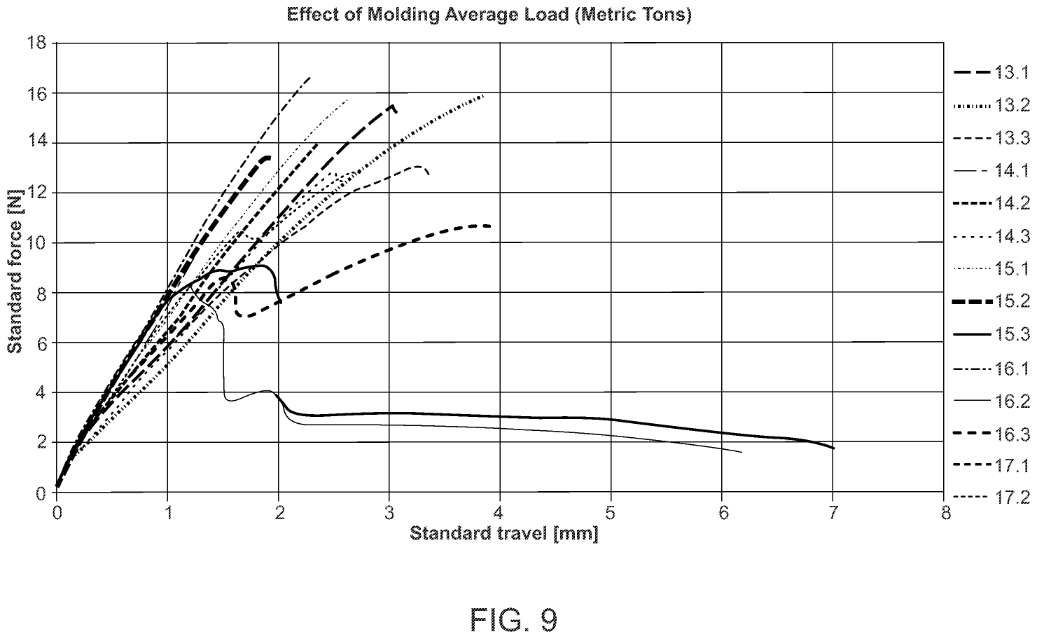

FIG. 9 shows stress strain curves generated from samples 13-17 to assess the effect of molding pressure (average load) on the mechanical properties of 18B solids. The 13, 14, 15, 16, and 17 series correspond to 1, 2, 3, 4, and 5 metric tons, respectively.

Flexural data for 18B solid samples at different average loads are shown in Table 9 below. Average values of flexural modulus (MPa), maximum flexural strength (MPa), and elongation at break (%) for each of the conditioned vs. unconditioned samples measured in triplicate (along with measured standard deviation (SD)) are provided.

TABLE 9
Effect of Molding Pressure on 18B Solid Samples (Flexural Data)

| | | Average | SD | Average Max. | SD Max. | Average | SD |
|---|---|---|---|---|---|---|---|
| | Average | Flexural | Flexural | Flexural | Flexural | Elongation | Elongation |
| Sample | Load | Modulus | Modulus | Strength | Strength | at Break | at Break |
| ID# | (metric tons) | (MPa) | (MPa) | (MPa) | (MPa) | (%) | (%) |
| 13 | 1 | 208.92 | 21.80 | 5.68 | 0.69 | 2.94 | 0.95 |
| 14 | 2 | 247.61 | 38.40 | 4.97 | 0.58 | 2.05 | 0.66 |
| 15 | 3 | 257.77 | 46.70 | 4.30 | 0.62 | 4.38 | 2.19 |
| 16 | 4 | 290.75 | 29.22 | 4.80 | 1.66 | 3.98 | 1.87 |
| 17 | 5 | 284.37 | 14.41 | 5.85 | 0.63 | 2.20 | 0.49 |

Sample ID #13-17 demonstrated the effect of different pressing loads for samples pressed for 5 minutes and cooled at a medium rate. The trend from increasing pressing load was an increase in flexural modulus, while the trend for strength and elongation percentage could not be confidently discerned due to variability. As set load increased, the strength was large when load averaged 1 metric ton (on average 5.68 MPa) before decreasing when load averaged between 2-4 metric tons and then increased to a maximum of 5.85 MPa when the average load was 5 metric tons. Still, the impact of press load on strength was inconclusive due to variability. Elongation percentage ranged between 2.05% to 4.38% depending on average load without any significant, noticeable trend. To maximize stiffness of the recombinant silk solid material, it was determined that an average load of 3-5 metric tons was preferred.

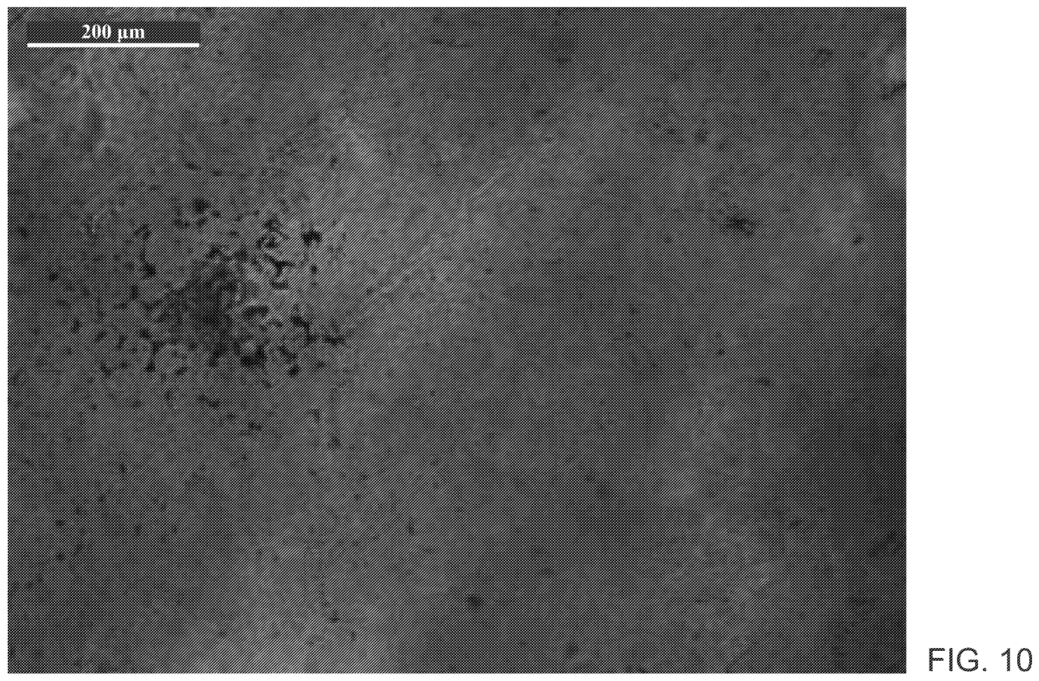

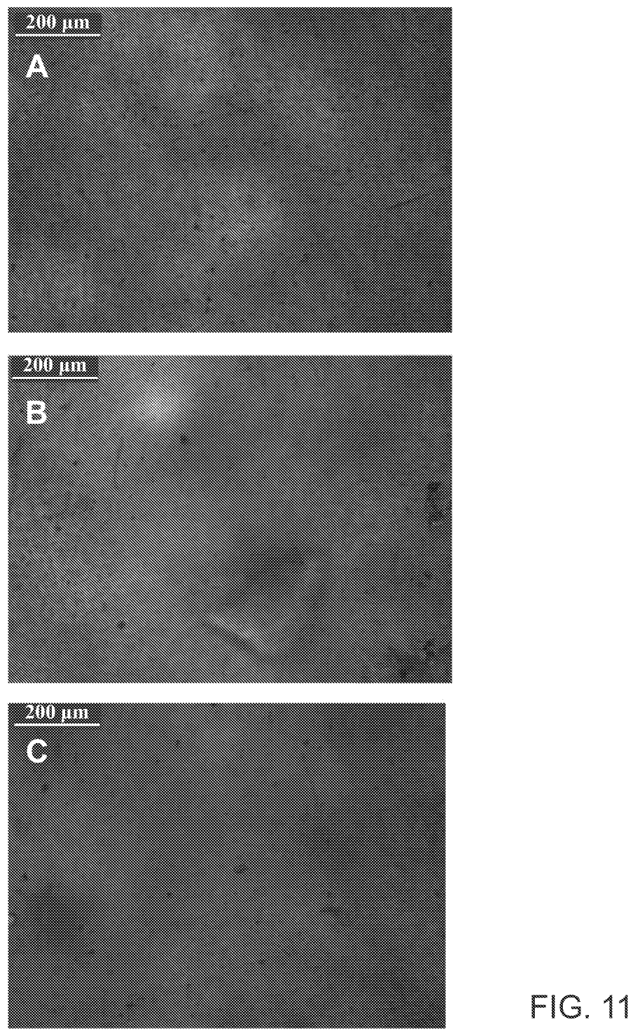

Dispersed protein particles appeared as black dots but depending on context may be porosity voids on surface as shown in FIG. 10. Particles tended to preferentially position themselves in these voids. Increasing pressing load appeared to reduce the number of dispersed particles, but the benefit diminished beyond 3 metric tons (as shown in FIG. 11). Specifically, FIG. 11 shows images of solids generated by different average pressing loads. There was a decrease in amount of dispersed protein particles as average load increased from (A) 1 metric ton to (B) 3 metric tons to (C) 5 metric tons.

Mold Time

18B solid samples were molded as described above using 4.0 g samples and molded at 130° C. under an average load of 2 metric tons. Samples were molded for 1, 2, 3, 4, 5, 6, 8, 10 or 15 minutes. Molded samples were cooled at a medium cooling rate and were not conditioned. The conditions for assessing the effect of post-mold conditioning are based on samples 2, 4, 6, 8, 14, 18, 19, 20 and 21 provided in Table 6 and Table 10.

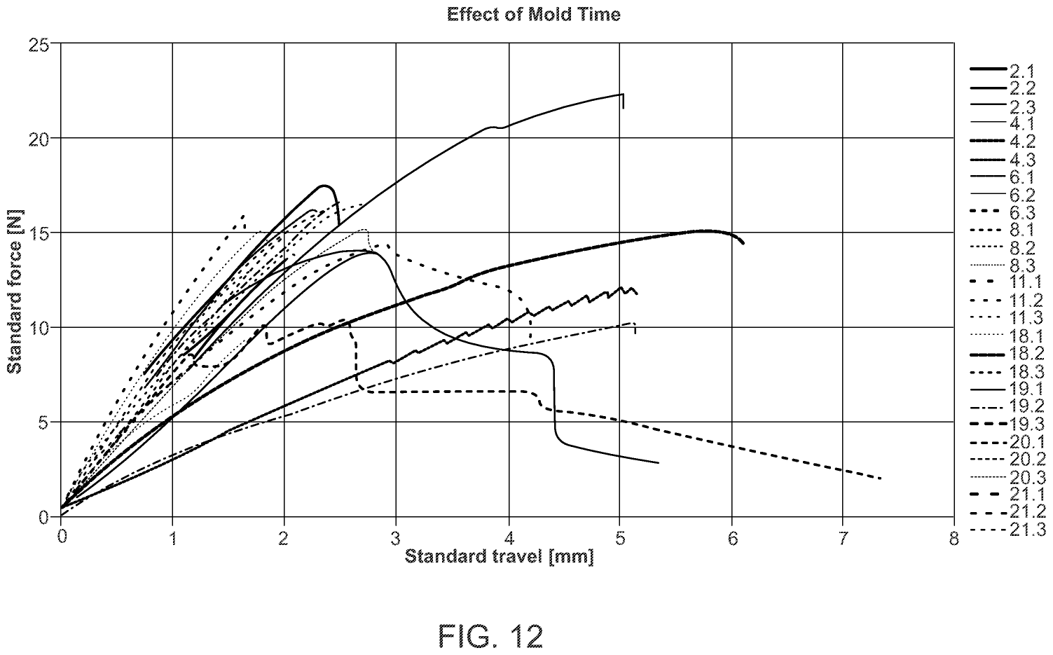

FIG. 12 shows stress-strain curves generated from samples 2, 4, 6, 8, 14, 18, 19, 20 and 21 to assess the effect of mold time on the mechanical properties of 18B solids. The 2, 4, 6, 8, 14, 18, 19, 20 and 21 series correspond with 1, 2, 3, 4, 5, 6, 8, 10 and 15 minute mold times, respectively.

Flexural data for 18B solid samples molded for different lengths of time are shown in Table 10 below. Average values of flexural modulus (MPa), maximum flexural strength (MPa), and elongation at break (%) for each of the conditioned vs. unconditioned samples measured in triplicate (along with measured standard deviation (SD)) are provided.

TABLE 10
Effect of Mold Time on 18B Solid Samples (Flexural Data)

| | | | | | | Average | SD |
|---|---|---|---|---|---|---|---|
| | | | | Average | SD | Elon- | Elon- |
| | | Average | SD | Max. | Max. | gation | gation |
| | Mold | Flexural | Flexural | Flexural | Flexural | at | at |
| Sample | Time | Modulus | Modulus | Strength | Strength | Break | Break |
| ID# | (min) | (MPa) | (MPa) | (MPa) | (MPa) | (%) | (%) |
| 2 | 1 | 193.44 | 77.14 | 4.51 | 0.32 | 2.97 | 1.86 |
| 4 | 2 | 220.12 | 155.59 | 4.93 | 0.99 | 3.19 | 1.81 |
| 6 | 3 | 252.80 | 18.00 | 5.39 | 2.51 | 2.79 | 2.13 |
| 8 | 4 | 255.06 | 65.23 | 5.61 | 0.34 | 4.70 | 1.84 |
| 14 | 5 | 247.61 | 38.40 | 4.97 | 0.58 | 2.05 | 0.66 |
| 18 | 6 | 263.83 | 41.24 | 5.34 | 0.90 | 2.23 | 0.48 |
| 19 | 8 | 305.70 | 21.77 | 5.76 | 0.93 | 2.19 | 0.17 |
| 20 | 10 | 323.16 | 79.54 | 4.44 | 1.04 | 3.61 | 3.22 |
| 21 | 15 | 346.06 | 16.78 | 5.25 | 1.32 | 1.99 | 0.16 |

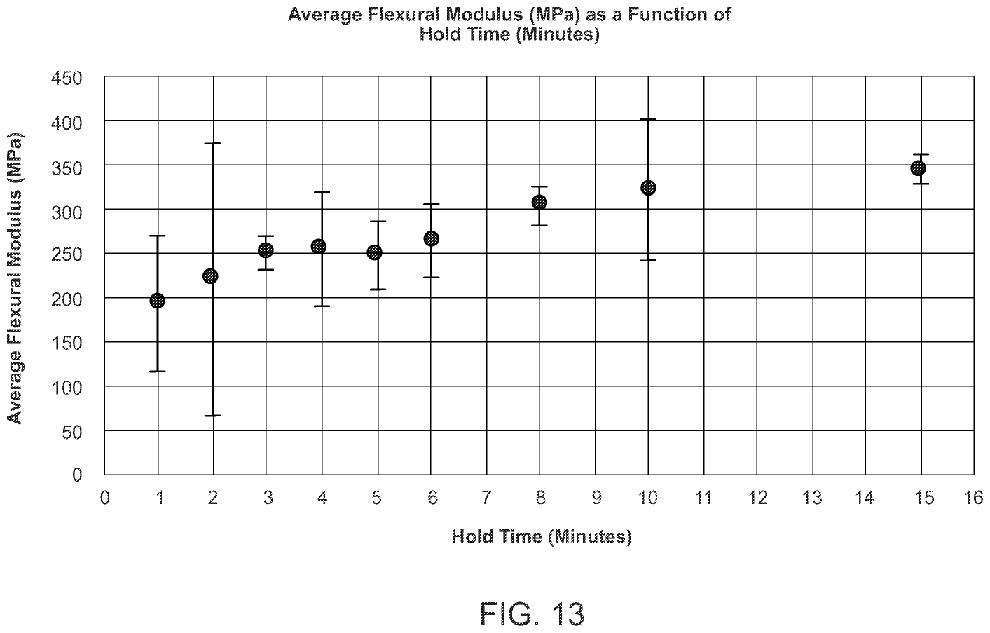

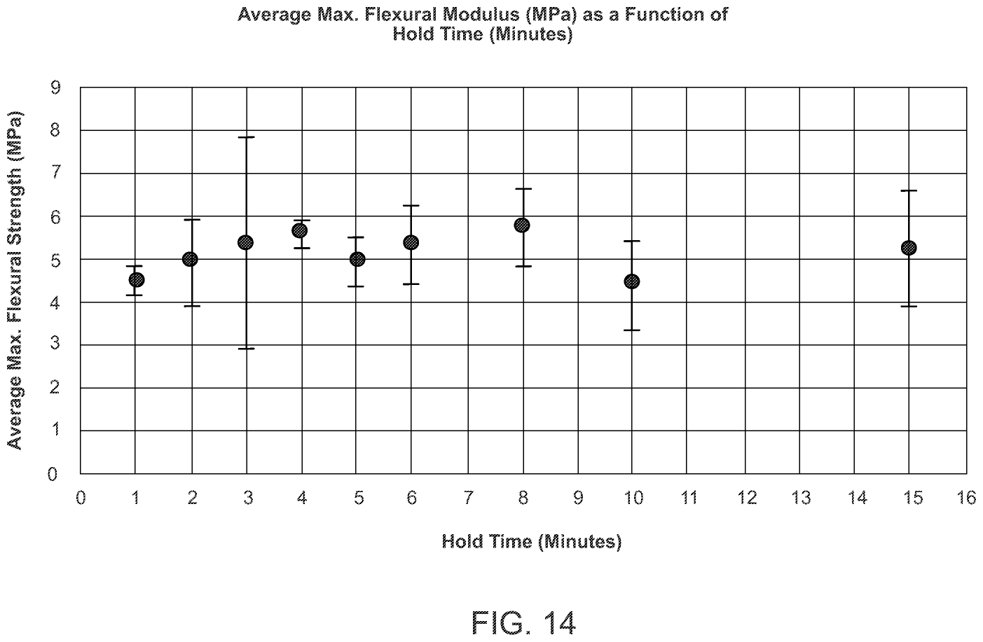

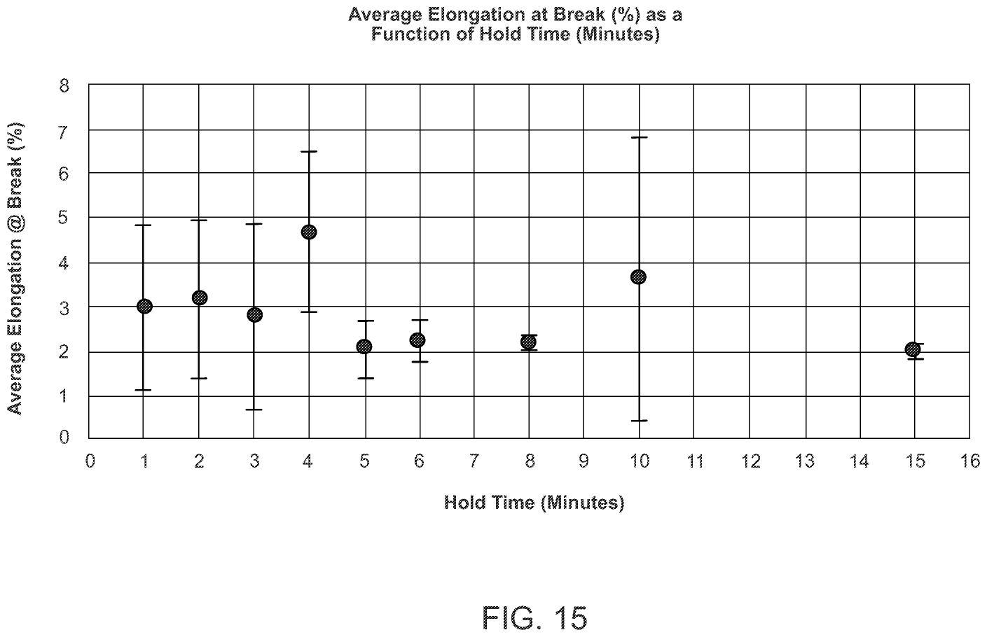

It was discovered that increasing molding hold time only suggested an increase in stiffness of the solid. There did not seem to be any statistically significant impact to the flexural strength and elongation percentage at break of the solid as mold time changed. This was supported in FIG. 13, FIG. 14, and FIG. 15 for average flexural modulus, average flexural strength, and average elongation at break, respectively. Specifically, FIG. 13 shows average flexural modulus (MPa) over holding time. As holding time increased, the average flexural modulus increased. Error bars show sample standard deviation. FIG. 14 shows average flexural strength (MPa) over holding time. There did not appear to be a statistically significant difference in maximum flexural strength across all molding times tested. FIG. 15 shows average elongation at break (%) over holding time. There did not appear to be any significant relationship between elongation percentage at break and holding time. Error bars are sample standard deviation.

Flexural modulus generally increased as hold time increased. Note that for flexural strength the nominal value for any given hold time was within the margin of error for the other mold times. For that reason, it could be concluded that there did not seem to be a significant difference in strength based on molding time. Similarly, there did not appear to be any significant relationship between holding time and elongation at break. Relatively large margins of error and variability can partially be explained by limiting testing to 3 specimens per sample group due to time constraints. From these results, it was recommended to center future processing around 5- to 8-minute mold times with 3-5 metric tons of average load and a medium cooling rate. While longer mold times could yield stiffer solids on average, increasing molding time too long resulted in a decrease in throughput/productivity. Alternatively, shorter mold times resulted in powder-like solids that were not exceptionally aesthetically pleasing.

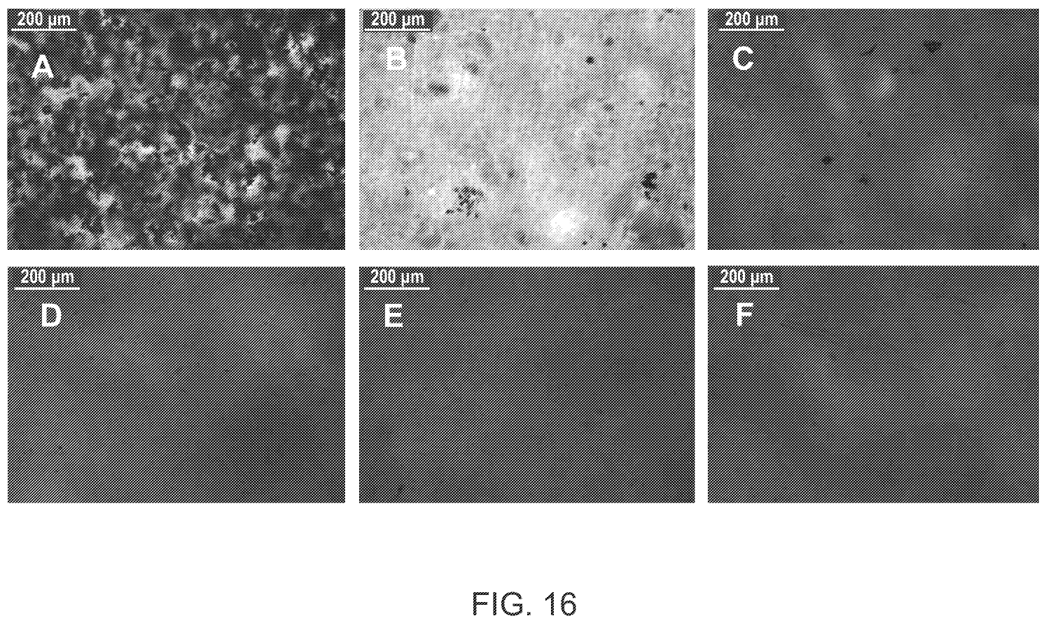

Optical light microscopy of the pre-fracture specimen surfaces was intended to reveal the effect of each of the four factors on solid morphology and to assist in understanding the role of each factor in solids processing. The results of varying only mold time from 1 minute to 15 minutes is shown in FIG. 16. Specifically, FIG. 16 shows the morphology of unconditioned solids subjected to various hold times maintaining equal average load and cooling rate: (A) 1 minute (B) 3 minutes (C) 5 minutes (D) 8 minutes (E) 10 minutes (F) 15 minutes. As mold time increased from 1 minute to 5 minutes, particle aggregates were greatly reduced with each additional minute of molding.

This conclusion was supported by visual macroscopic examination as shown in FIG. 17 where longer mold times led to more homogenous, translucent solids. Specifically, FIG. 17 shows a macroscopic visual examination between 1-minute hold time and 5-minute hold time against (A) solid black surface (B, C) bright light. Solids with longer hold times yielded fewer noticeable powder clumps and were more translucent. There was a noticeable lack of significant differentiation beyond 5-6 minutes, though particle aggregates were still present even at 15 minutes. A recommended mold time was 5 minutes for thicknesses ranging up to 3 mm, to avoid exposing the protein to elevated temperatures for prolonged durations and to minimize noticeable particle aggregates.

FIG. 18 shows a post-fracture surface of the recombinant silk molded body imaged with Benchtop SEM across different mold times. (A) 1-minute hold time darkened for greater contrast (B) 5-minute mold time (C) 15-minute mold time. The 5-minute hold time showed the greatest mix of ductility and brittle behavior.

### CONCLUSIONS

The specimens with the greatest stiffnesses were a result of higher molding times and increased pressing loads. It was recommended to explore these samples as the best path forward for stiff solids. The most promising specimens were from Sample ID #11, 12, and 17. Strength and elongation trends based on molding time could not be confidently discerned due to high sample to sample variation.

A recommended mold time was between 5 to 8 minutes. While longer mold times could yield stiffer solids on average, increasing molding time too long resulted in a decrease in throughput/productivity and caused protein degradation. Alternatively, shorter mold times below 5 minutes resulted in powder-like solids that were not exceptionally aesthetically pleasing.

There did not appear to be a statistically significant difference in moduli, maximum strength, and elongation at break between fast, medium, and slow cooling. Because medium and slow cooling were most convenient to implement, they were recommended.

The specimens with the greatest elongation percentage at break were conditioned in 65% relative humidity (RH) for a minimum of 72 hours and demonstrated elongation percentages at break far beyond the capabilities of the Zwick ProLine apparatus.

### Example 4: Cross-Linked Recombinant Silk Solids

18B solids were cross-linked using ammonium persulfate. Ammonium persulfate dissolved in water but did not dissolve in TEOA or IPA. Water had negative effects on making the solid, and the solid could not be left in water for prolonged times as it swelled and disintegrated. However, it was possible to dissolve ammonium persulfate in water and mix it with another solvent.

Two ways were attempted to use ammonium persulfate to cross link the solid. In the first method, 79.7 mg of ammonium persulfate (APS) was added to 100.4 mg of water and dissolved using vortex mixer. The solution was added to 7.79 g of TEOA and mixed using the vortex mixer. This resulted in a 50 mM solution of ammonium persulfate in 99/1 TEOA/water solution.

The solution was dispersed in 9.518 g of 18B, resulting in a 55% by weight 18B dispersion. The mixture was placed in a mold and pressed at 130-135° C. The solid was left in the oven to cure for 15 hours and then placed in water. The solid swelled and started to disintegrate in water indicating that cross linking did not take place.

In another cross-linking method, the 18B pressed solid was immersed in ammonium persulfate (APS) solution. 684 mg of APS was dissolved in 1.3 mL of DI water. Since the solid swelled excessively and disintegrated in pure water, IPA was added to the solution. Adding 11.45 mL of IPA resulted in the APS crashing out of the solution. Upon adding another 3.3 mL of water the salt went back into solution, resulting in a 187 mM APS solution in 71/29 IPA/water mixture. In terms of weight percent, there was 5 wt % of ammonium persulfate, 32 wt % of water and 63 wt % of water.

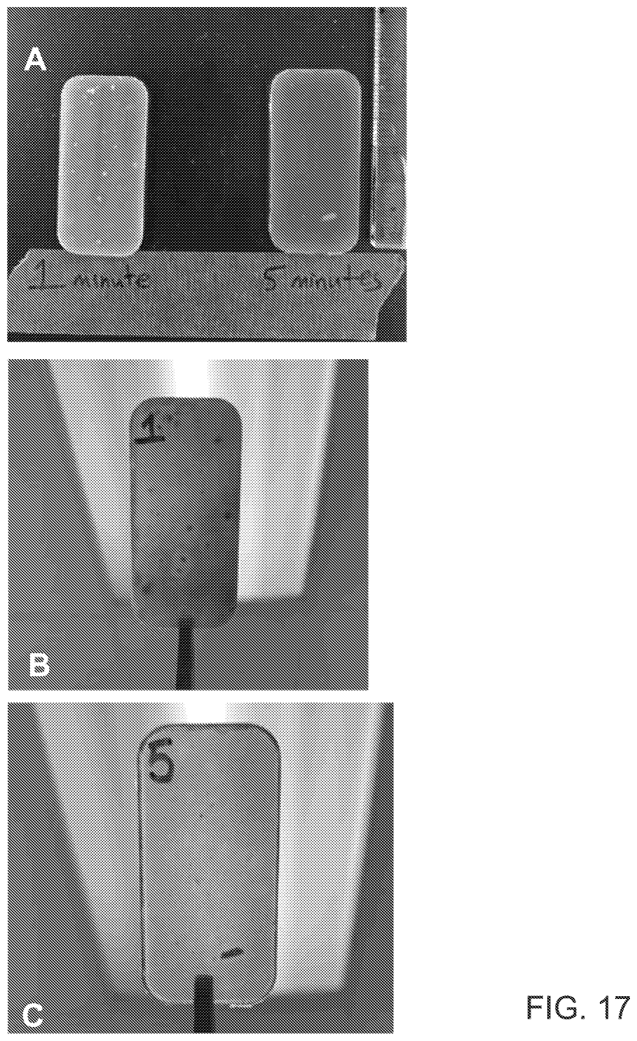

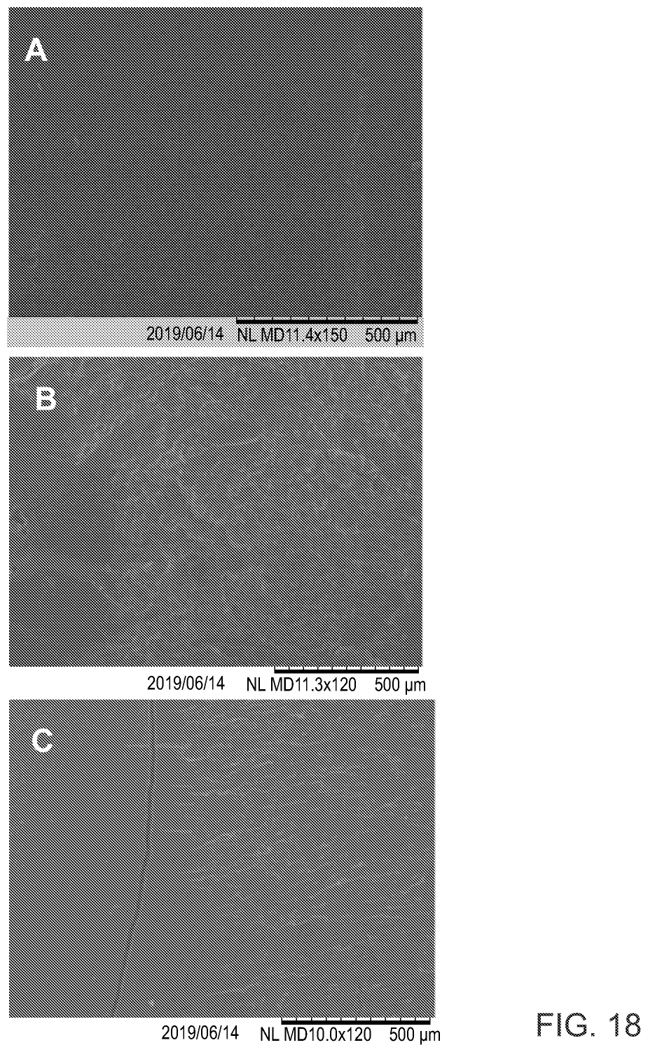

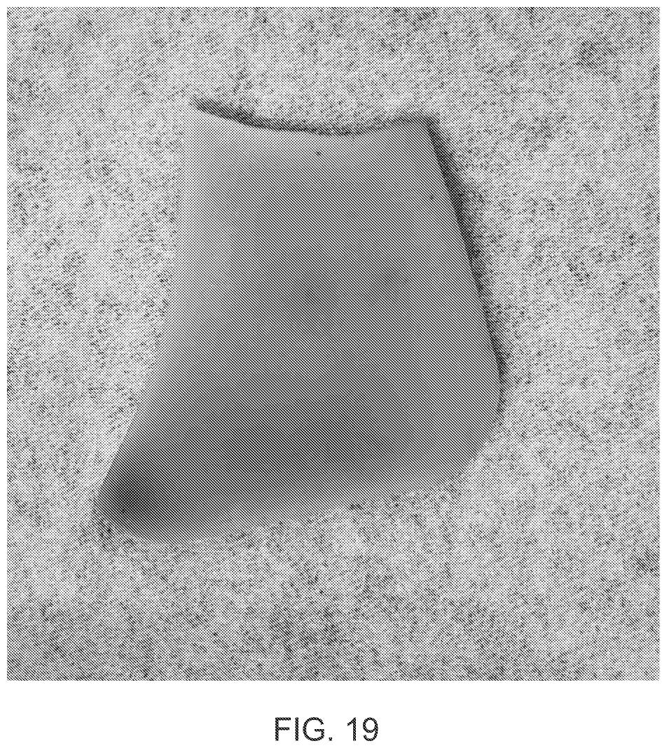

TEOA pressed sample was immersed in the cross linking solution for 1 hour and then stored at 80° C. for 3 hours. The resulting solid was water resistant and would not disintegrate in water even after 1 day of water exposure (FIG. 19).

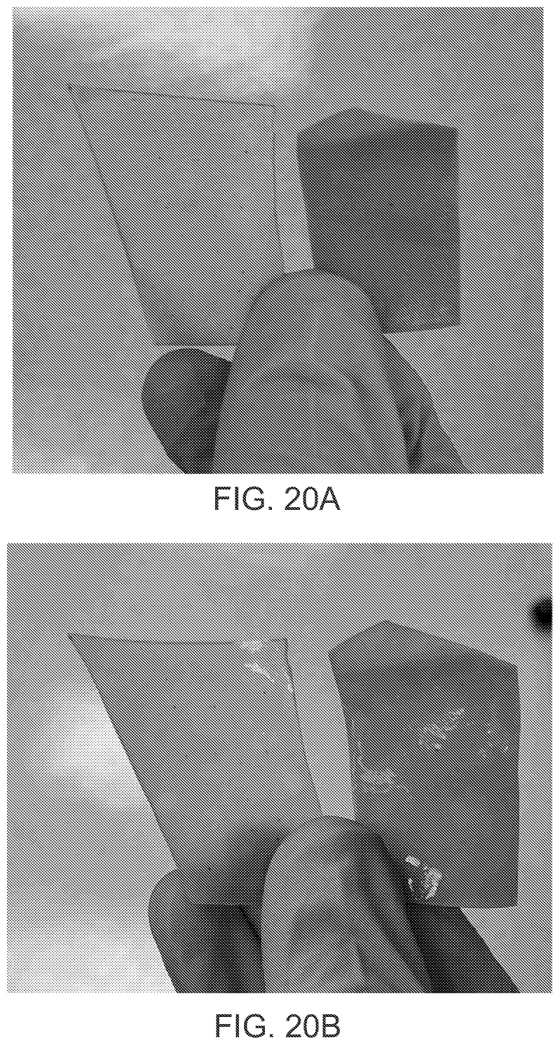

Cross linking was carried out for glycerol pressed films as well. The films were soaked in the APS/IPA/water solution for 10 and 60 minutes and cured overnight. The film that was soaked for a longer time was more opaque, especially when wetted. After curing in the oven, the dried films were rigid and brittle (FIG. 20A). After soaking in water for less than an hour, water diffused in the structure resulting in rubbery behavior (FIG. 20B).

In addition to water resistance, cross linking also resolved another issue with the solid materials. As plasticizers are all hygroscopic, the solids take up water and lose dimensional stability. Solid pressed samples held at high humidity levels become soft and flexible, similar to the glycerol pressed films. Cross linking helped maintain the structural integrity of the materials. Solids pressed with 10 wt % propanediol at 130° C. were cross linked using two chemistries, glutaraldehyde and ammonium persulfate.

The glutaraldehyde chemistry consisted of 10 wt % glutaraldehyde, 10 wt % water, 1.5 wt % aluminum chloride hexahydrate and 78.5 wt % isopropyl alcohol. Solids were left soaking in the cross linking solution for 12 hours and then placed in a hot oven at 125° C. for 5 minutes for curing.

The ammonium persulfate chemistry consisted of 5 wt % ammonium persulfate, 25 wt % of water and 73 wt % isopropyl alcohol. The solid was placed in the chemistry for 1 hour and placed at 60° C. for 3 hours for curing.

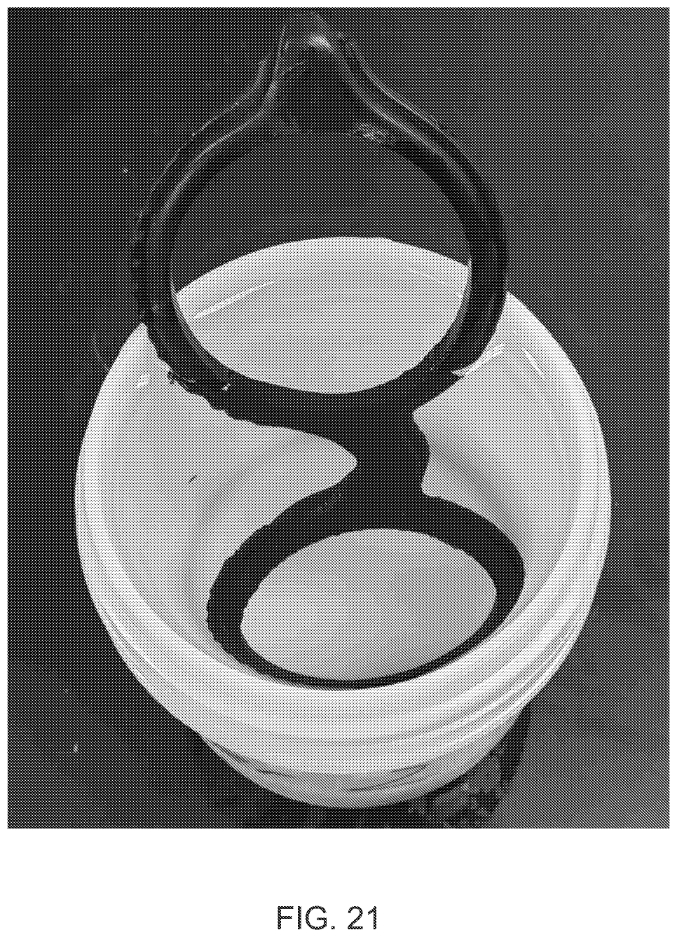

After cross linking with either chemistry, the solids became water resistant and retained their shape when immersed in water (FIG. 21).

### Example 5: Formation of Films from Recombinant Silk Protein

Film Pressing

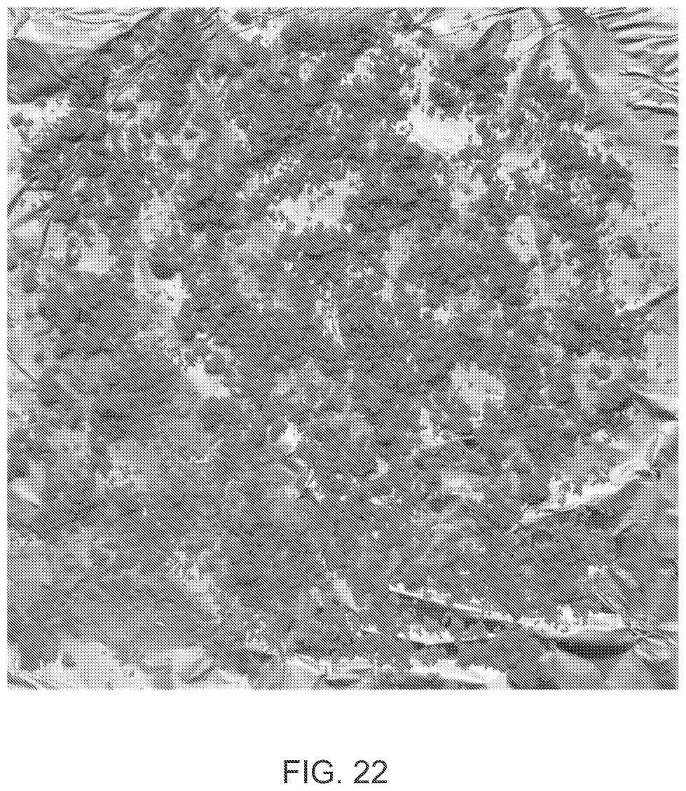

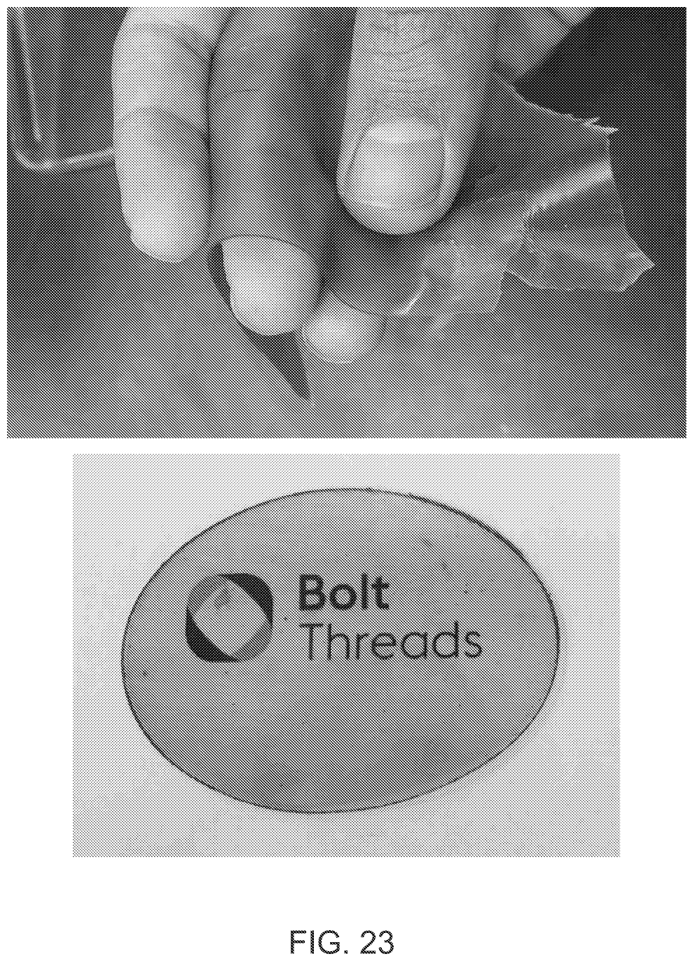

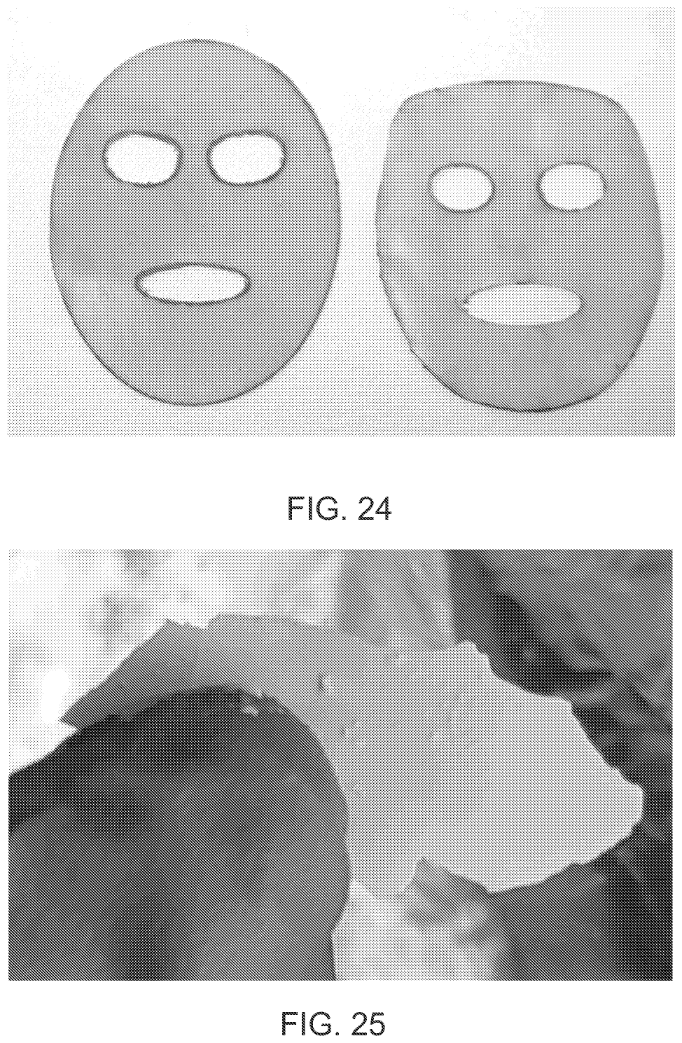

Solvated 18B powder in 30-50% by weight glycerol as the plasticizer was also dispersed onto a surface (FIG. 22) and pressed between two parallel plates with glycerol. Films pressed with glycerol easily bended and could conform to surfaces, while the other solvents formed rigid and brittle film. The drapability increased as the film thickness decreased. These flexible films were optically transparent (FIG. 23). These films could be cut using a laser cutter or using dies (FIG. 24).

As control, 18B was pressed at 130° C. without any solvents, resulting in a brittle film white film (FIG. 25), where the powder was simply flattened and compacted into a film.

Film Extrusion

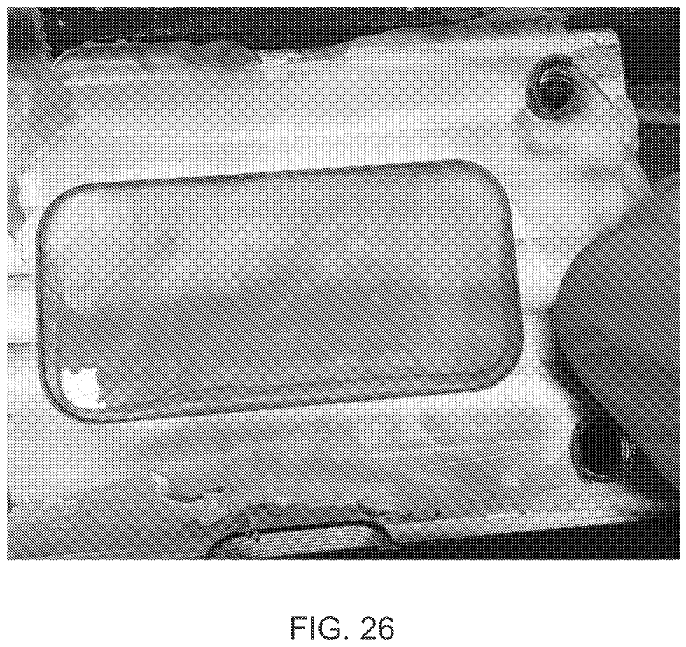

Solvated 18B was extruded as an 18B film extrusion. During pressing to form an 18B solid/film described in examples 1 and 2, dope flowed between the flush surfaces, referred to as flash, and formed a thin flexible film (FIG. 26). Thus, film formation was performed through extrusion.

### Example 6: Re-Molding of Recombinant Silk Solids

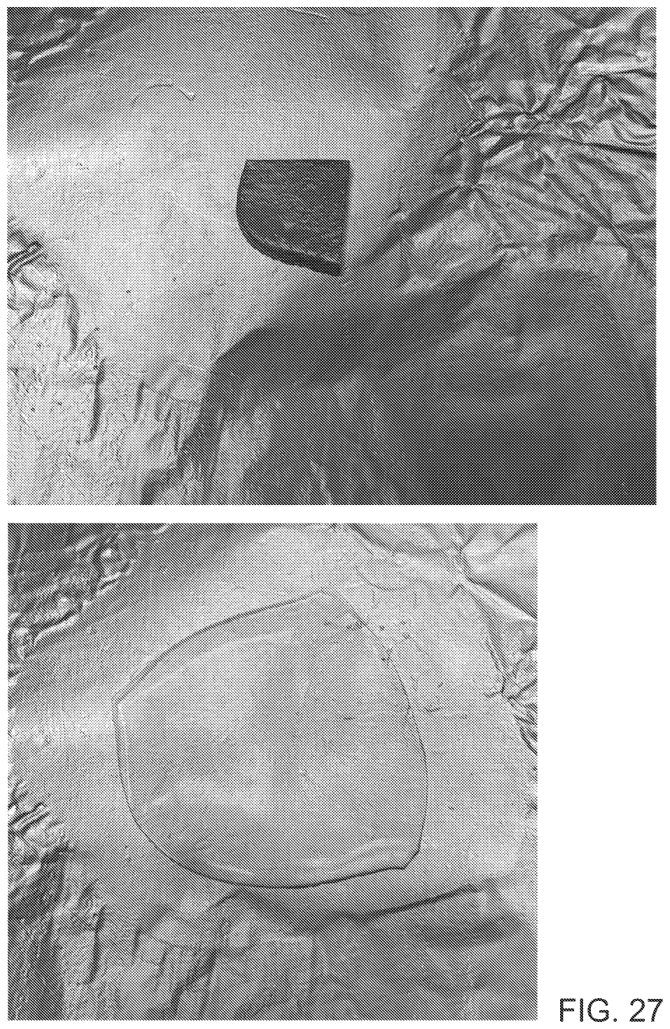

A molded 18B solid prepared by pressing with 1,3 propanediol as described in Example 1 was reprocessed and pressed at 130° C. to form a thin film. A photograph of the reprocessed film is shown in FIG. 27. Specifically, the original 18B solid prepared by pressing with 1,3 propanediol is on the left, and the re-processed film is shown on the right. This result indicates that the recombinant silk solids described herein can be re-processed using the methods described herein to form different molded body shapes.

### OTHER EMBODIMENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method for preparing a molded body, comprising:
a. providing a composition comprising recombinant silk and plasticizer, wherein said composition is in a flowable state;
b. placing said composition in a mold;
c. applying heat and pressure to said composition in said mold; and
d. cooling said composition to form a molded body comprising said recombinant silk.

2. The method of claim 1, wherein said molded body is in a solid form.

3. The method of claim 1, wherein said molded body is a film.

4. The method of claim 1, wherein the recombinant silk is a recombinant silk powder distributed in said plasticizer.

5. The method of claim 1, wherein said recombinant silk comprises a crystallinity similar to or less than the crystallinity of 18B before molding.

6. The method of claim 1, wherein said recombinant silk protein is Nephila spider flagelliform silk or Araneus spider silk.

7. The method of claim 1, wherein said recombinant silk is 18B.

8. The method of claim 1, wherein said recombinant silk comprises SEQ ID NO: 1.

9. The method of claim 1, wherein said plasticizer is selected from the group consisting of: triethanolamine, trimethylene glycol, or propylene glycol.

10. The method of claim 1, wherein said composition comprises 15% by weight trimethylene glycol.

11. The method of claim 1, wherein said plasticizer is from 10-50% by weight of said composition.

12. The method of claim 1, wherein said heat is applied at a temperature of 130° C.

13. The method of claim 1, wherein said pressure is applied in the range of 1,500 to 15,000 psi.

14. The method of claim 1, wherein said molded body has a hardness of 100 as measured by a Type A durometer.

15. The method of claim 1, wherein said molded body has a hardness 90 or more as measured by a Type A duromoter.

16. The method of claim 1, wherein said molded body has a hardness 50 or more, 60 or more, or 70 or more as measured by a Type D durometer.

17. The method of claim 1, wherein said molded body can be machined, cut, or drilled and maintain its desired shape.

18. The method of claim 1, wherein said molded body has at least 50%, 60%, 70%, 80%, or 90% full length 18B monomers as compared to the recombinant silk of said composition in said flowable state.

19. The method of claim 1, wherein said molded body has at least 35%, at least 40%, at least 45%, or at least 50% full length recombinant silk monomers.

20. The method of claim 1, wherein said molded body has at least 50% total recombinant silk monomers, recombinant silk aggregates, and high molecular weight intermediates.

21. The method of claim 1, wherein said heat and pressure is applied for minute, 2 minutes, 3 minutes, 4 minutes, 5 minutes, 6 minutes, 8 minutes, 10 minutes, or 15 minutes.

22. The method of claim 1, wherein said heat and pressure is applied for from 5 to 8 minutes.

23. The method of claim 1, further comprising exposing said molded body to a relative humidity of at least 50% for at least 24 hours.

24. The method of claim 1, further comprising exposing said molded body to a relative humidity of 65% for 72 hours.

25. The method of claim 1, wherein said pressure is applied by a pressing load of at least 1 metric ton, at least 2 metric tons, at least at least 3 metric tons, at least 4 metric tons, or at least 5 metric tons.

26. The method of claim 1, wherein said pressure is applied by a pressing load from 1 to 5 metric tons, or from 3 to 5 metric tons.

27. The method of claim 1, wherein said cooling is at a rate of about 1° C./min, about 3° C./min, or about 45° C./min.

28. The method of claim 1, wherein said composition has a flexural modulus of 50 MPa or more, 60 MPa or more, 70 MPa or more, 80 MPa or more, 90 MPa or more, 100 MPa or more, 150 MPa or more, 200 MPa or more, 250 MPa or more, or 300 MPa or more.

29. The method of claim 1, wherein said composition has a maximum flexural strength of 10 MPa or more, 20 MPa or more, 30 MPa or more, 40 MPa or more, 50 MPa or more, 60 MPa or more, 70 MPa or more, 80 MPa or more MPa or more, 90 MPa or more or 100 MPa or more.

30. The method of claim 1, wherein said composition has an elongation percentage at break of 1 to 4%.

31. The method of claim 1, wherein said composition has an elongation percentage at break of greater than 20%.

32. The method of claim 1, wherein said composition further comprises ammonium persulfate.

33. The method of claim 1, further comprising immersing said molded body in ammonium persulfate.

34. The method of claim 1, wherein said molded body is cross-linked.

35. The method of claim 1, wherein said molded body is a cosmetic or skincare formulation.

36. A composition comprising a recombinant silk and a plasticizer, wherein said composition is in a solid form.

37. The composition of claim 36, wherein said molded body is in a solid form.

38. The composition of claim 36, wherein said molded body is a film.

39. The composition of claim 36, wherein the recombinant silk is a recombinant silk powder distributed in said plasticizer.

40. The composition of claim 36, wherein said recombinant silk is 18B.

41. The composition of claim 36, wherein said recombinant silk comprises SEQ ID NO: 1.

42. The composition of claim 36, wherein said plasticizer is selected from the group consisting of: triethanolamine, trimethylene glycol, or propylene glycol.

43. The composition of claim 36, wherein said composition comprises 15% by weight trimethylene glycol.

44. The composition of claim 36, wherein said plasticizer is from 10-50% by weight of said composition.

45. The composition of claim 36, wherein said molded body has a hardness of 100 as measured by a Type A durometer.

46. The composition of claim 36, wherein said molded body has a hardness 90 or more as measured by a Type A duromoter.

47. The composition of claim 36, wherein said molded body has a hardness 50 or more, 60 or more, or 70 or more as measured by a Type D durometer.

48. The composition of claim 36, wherein said molded body can be machined, cut, or drilled and maintain its desired shape.

49. The composition of claim 36, wherein said molded body has at least 50%, 60%, 70%, 80%, or 90% full length 18B monomers as compared to the recombinant silk of said composition in said flowable state.

50. The composition of claim 36, wherein said molded body has at least 35%, at least 40%, at least 45%, or at least 50% full length recombinant silk monomers.

51. The composition of claim 36, wherein said molded body has at least 50% total recombinant silk monomers, recombinant silk aggregates, and high molecular weight intermediates.

52. The composition of claim 36, wherein said composition has a flexural modulus of 50 MPa or more, 60 MPa or more, 70 MPa or more, 80 MPa or more, 90 MPa or more, 100 MPa or more, 150 MPa or more, 200 MPa or more, 250 MPa or more, or 300 MPa or more.

53. The composition of claim 36, wherein said composition has a maximum flexural strength of 10 MPa or more, 20 MPa or more, 30 MPa or more, 40 MPa or more, 50 MPa or more, 60 MPa or more, 70 MPa or more, 80 MPa or more MPa or more, 90 MPa or more or 100 MPa or more.

54. The composition of claim 36, wherein said composition has an elongation percentage at break of 1 to 4%.

55. The composition of claim 36, wherein said composition has an elongation percentage at break of greater than 20%.

56. The composition of claim 36, wherein said composition further comprises ammonium persulfate.

57. The composition of claim 36, wherein said molded body is cross-linked.

58. The composition of claim 36, wherein said molded body is a cosmetic or skincare formulation.


SEQUENCE LISTING (38 sequences)

SEQ ID NO: 1 - protein, 945 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 2 - protein, 315 aa, Artificial Sequence
  Description of Artificial Sequence Synthetic polypeptide
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 3 - protein, 5 aa, Unknown
  Description of Unknown Silk polypeptide block sequence
SGAGG

SEQ ID NO: 4 - protein, 5 aa, Unknown
  Description of Unknown Silk polypeptide block sequence
GSGAG

SEQ ID NO: 5 - protein, 5 aa, Unknown
  Description of Unknown Silk polypeptide block sequence
GGSGA

SEQ ID NO: 6 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 7 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 8 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 9 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 10 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 11 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 12 - protein, 171 aa, Nephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 13 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 14 - protein, 420 aa, Nephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 15 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 16 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 17 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 18 - protein, 32 aa, Euprosthenops australis
GGQGGQGQGRYGQGAGSSAAAAAAAAAAAAAA

SEQ ID NO: 19 - protein, 42 aa, Tetragnatha kauaiensis
GGLGGGQGAGQGGQQGAGQGGYGSGLGGAGQGASAAAAAAAA

SEQ ID NO: 20 - protein, 42 aa, Argiope aurantia
GGYGPGAGQQGPGSQGPGSGGQQGPGGLGPYGPSAAAAAAAA

SEQ ID NO: 21 - protein, 46 aa, Deinopis spinosa
GPGGYGGPGQQGPGQGQYGPGTGQQGQGPSGQQGPAGAAAAAAAAA

SEQ ID NO: 22 - protein, 42 aa, Nephila clavata
GPGGYGLGQQGPGQQGPGQQGPAGYGPSGLSGPGGAAAAAAA

SEQ ID NO: 23 - protein, 174 aa, Deinopis spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 24 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 25 - protein, 161 aa, Nephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 26 - protein, 186 aa, Nephilengys cruentata
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 27 - protein, 132 aa, Uloborus diversus
GSGAGAGSGYGAGAGAGAGSGYGAGSSASAGSAINTQTVTSSTTTSSQSSAAATGAGYGTGAGTGASAGAAASGAGAGYGGQAGYGQGAGASARAAGSGYGAGAGAAAAAGSGYGAGAGAGAGSGYGAGAAA

SEQ ID NO: 28 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 29 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 30 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 31 - protein, 177 aa, Nephilengys cruentata
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 32 - protein, 174 aa, Nephilengys cruentata
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 33 - protein, 8 aa, Artificial Sequence
  Description of Artificial Sequence Synthetic His tag
  SITE (1)..(8): This sequence may encompass 6-8 residues
HHHHHHHH

SEQ ID NO: 34 - protein, 1600 aa, Artificial Sequence
  Description of Artificial Sequence Synthetic polypeptide
  SITE (1)..(1600): This sequence may encompass 2-20 “GGY-[GPG-X1]n1-GPS-(A)n2” repeating units, wherein X1 is “SGGQQ,” “GAGQQ,” “GQGPY,” “AGQQ” or “SQ,” n1 is 4-8 and n2 is 6-10 and some positions may be absent
  SITE (4)..(67): This region may encompass 4-8 repeating “GPG-X1” repeating units, wherein X1 is “SGGQQ,” “GAGQQ,” “GQGPY,” “AGQQ” or “SQ,” and some positions may be absent
  SITE (7)..(11): This region may encompass “SGGQQ,” “GAGQQ,” “GQGPY,” “AGQQ” or “SQ,” wherein some positions may be absent
  SITE (15)..(19): This region may encompass “SGGQQ,” “GAGQQ,” “GQGPY,” “AGQQ” or “SQ,” wherein some positions may be absent
  SITE (23)..(27): This region may encompass “SGGQQ,” “GAGQQ,” “GQGPY,” “AGQQ” or “SQ,” wherein some positions may be absent
  SITE (31)..(35): This region may encompass “SGGQQ,” “GAGQQ,” “GQGPY,” “AGQQ” or “SQ,” wherein some positions may be absent
  SITE (39)..(43): This region may encompass “SGGQQ,” “GAGQQ,” “GQGPY,” “AGQQ” or “SQ,” wherein some positions may be absent
  SITE (47)..(51): This region may encompass “SGGQQ,” “GAGQQ,” “GQGPY,” “AGQQ” or “SQ,” wherein some positions may be absent
  SITE (55)..(59): This region may encompass “SGGQQ,” “GAGQQ,” “GQGPY,” “AGQQ” or “SQ,” wherein some positions may be absent
  SITE (63)..(67): This region may encompass “SGGQQ,” “GAGQQ,” “GQGPY,” “AGQQ” or “SQ,” wherein some positions may be absent
  SITE (71)..(80): This region may encompass 6-10 residues
  SITE (84)..(147): This region may encompass 4-8 repeating “GPG-X1” repeating units, wherein X1 is “SGGQQ,” “GAGQQ,” “GQGPY,” “AGQQ” or “SQ,” and some positions may be absent
  SITE (87)..(91): This region may encompass “SGGQQ,” “GAGQQ,” “GQGPY,” “AGQQ” or “SQ,” wherein some positions may be absent
  SITE (95)..(99): This region may encompass “SGGQQ,” “GAGQQ,” “GQGPY,” “AGQQ” or “SQ,” wherein some positions may be absent
  SITE (103)..(107): This region may encompass “SGGQQ,” “GAGQQ,” “GQGPY,” “AGQQ” or “SQ,” wherein some positions may be absent
  SITE (111)..(115): This region may encompass “SGGQQ,” “GAGQQ,” “GQGPY,” “AGQQ” or “SQ,” wherein some positions may be absent
  SITE (119)..(123): This region may encompass “SGGQQ,” “GAGQQ,” “GQGPY,” “AGQQ” or “SQ,” wherein some positions may be absent
  SITE (127)..(131): This region may encompass “SGGQQ,” “GAGQQ,” “GQGPY,” “AGQQ” or “SQ,” wherein some positions may be absent
  SITE (135)..(139): This region may encompass “SGGQQ,” “GAGQQ,” “GQGPY,” “AGQQ” or “SQ,” wherein some positions may be absent
  SITE (143)..(147): This region may encompass “SGGQQ,” “GAGQQ,” “GQGPY,” “AGQQ” or “SQ,” wherein some positions may be absent
  SITE (151)..(160): This region may encompass 6-10 residues
  SITE (164)..(227): This region may encompass 4-8 repeating “GPG-X1” repeating units, wherein X1 is “SGGQQ,” “GAGQQ,” “GQGPY,” “AGQQ” or “SQ,” and some positions may be absent
  SITE (167)..(171): This region may encompass “SGGQQ,” “GAGQQ,” “GQGPY,” “AGQQ” or “SQ,” wherein some positions may be absent
  SITE (175)..(179): This region may encompass “SGGQQ,” “GAGQQ,” “GQGPY,” “AGQQ” or “SQ,” wherein some positions may be absent
  SITE (183)..(187): This region may encompass “SGGQQ,” “GAGQQ,” “GQGPY,” “AGQQ” or “SQ,” wherein some positions may be absent
  SITE (191)..(195): This region may encompass “SGGQQ,” “GAGQQ,” “GQGPY,” “AGQQ” or “SQ,” wherein some positions may be absent
  SITE (199)..(203): This region may encompass “SGGQQ,” “GAGQQ,” “GQGPY,” “AGQQ” or “SQ,” wherein some positions may be absent
  SITE (207)..(211): This region may encompass “SGGQQ,” “GAGQQ,” “GQGPY,” “AGQQ” or “SQ,” wherein some positions may be absent
  SITE (215)..(219): This region may encompass “SGGQQ,” “GAGQQ,” “GQGPY,” “AGQQ” or “SQ,” wherein some positions may be absent
  SITE (223)..(227): This region may encompass “SGGQQ,” “GAGQQ,” “GQGPY,” “AGQQ” or “SQ,” wherein some positions may be absent
  SITE (231)..(240): This region may encompass 6-10 residues
  SITE (244)..(307): This region may encompass 4-8 repeating “GPG-X1” repeating units, wherein X1 is “SGGQQ,” “GAGQQ,” “GQGPY,” “AGQQ” or “SQ,” and some positions may be absent
  SITE (247)..(251): This region may encompass “SGGQQ,” “GAGQQ,” “GQGPY,” “AGQQ” or “SQ,” wherein some positions may be absent
  SITE (255)..(259): This region may encompass “SGGQQ,” “GAGQQ,” “GQGPY,” “AGQQ” or “SQ,” wherein some positions may be absent
  SITE (263)..(267): This region may encompass “SGGQQ,” “GAGQQ,” “GQGPY,” “AGQQ” or “SQ,” wherein some positions may be absent
  SITE (271)..(275): This region may encompass “SGGQQ,” “GAGQQ,” “GQGPY,” “AGQQ” or “SQ,” wherein some positions may be absent
  SITE (279)..(283): This region may encompass “SGGQQ,” “GAGQQ,” “GQGPY,” “AGQQ” or “SQ,” wherein some positions may be absent
  SITE (287)..(291): This region may encompass “SGGQQ,” “GAGQQ,” “GQGPY,” “AGQQ” or “SQ,” wherein some positions may be absent
  SITE (295)..(299): This region may encompass “SGGQQ,” “GAGQQ,” “GQGPY,” “AGQQ” or “SQ,” wherein some positions may be absent
  SITE (303)..(307): This region may encompass “SGGQQ,” “GAGQQ,” “GQGPY,” “AGQQ” or “SQ,” wherein some positions may be absent
  SITE (311)..(320): This region may encompass 6-10 residues
  SITE (324)..(387): This region may encompass 4-8 repeating “GPG-X1” repeating units, wherein X1 is “SGGQQ,” “GAGQQ,” “GQGPY,” “AGQQ” or “SQ,” and some positions may be absent
  SITE (327)..(331): This region may encompass “SGGQQ,” “GAGQQ,” “GQGPY,” “AGQQ” or “SQ,” wherein some positions may be absent
  SITE (335)..(339): This region may encompass “SGGQQ,” “GAGQQ,” “GQGPY,” “AGQQ” or “SQ,” wherein some positions may be absent
  SITE (343)..(347): This region may encompass “SGGQQ,” “GAGQQ,” “GQGPY,” “AGQQ” or “SQ,” wherein some positions may be absent
  SITE (351)..(355): This region may encompass “SGGQQ,” “GAGQQ,” “GQGPY,” “AGQQ” or “SQ,” wherein some positions may be absent
  SITE (359)..(363): This region may encompass “SGGQQ,” “GAGQQ,” “GQGPY,” “AGQQ” or “SQ,” wherein some positions may be absent
  SITE (367)..(371): This region may encompass “SGGQQ,” “GAGQQ,” “GQGPY,” “AGQQ” or “SQ,” wherein some positions may be absent
  SITE (375)..(379): This region may encompass “SGGQQ,” “GAGQQ,” “GQGPY,” “AGQQ” or “SQ,” wherein some positions may be absent
  SITE (383)..(387): This region may encompass “SGGQQ,” “GAGQQ,” “GQGPY,” “AGQQ” or “SQ,” wherein some positions may be absent
  SITE (391)..(400): This region may encompass 6-10 residues
  SITE (404)..(467): This region may encompass 4-8 repeating “GPG-X1” repeating units, wherein X1 is “SGGQQ,” “GAGQQ,” “GQGPY,” “AGQQ” or “SQ,” and some positions may be absent
  SITE (407)..(411): This region may encompass “SGGQQ,” “GAGQQ,” “GQGPY,” “AGQQ” or “SQ,” wherein some positions may be absent
  SITE (415)..(419): This region may encompass “SGGQQ,” “GAGQQ,” “GQGPY,” “AGQQ” or “SQ,” wherein some positions may be absent
  SITE (423)..(427): This region may encompass “SGGQQ,” “GAGQQ,” “GQGPY,” “AGQQ” or “SQ,” wherein some positions may be absent
  SITE (431)..(435): This region may encompass “SGGQQ,” “GAGQQ,” “GQGPY,” “AGQQ” or “SQ,” wherein some positions may be absent
  SITE (439)..(443): This region may encompass “SGGQQ,” “GAGQQ,” “GQGPY,” “AGQQ” or “SQ,” wherein some positions may be absent
  SITE (447)..(451): This region may encompass “SGGQQ,” “GAGQQ,” “GQGPY,” “AGQQ” or “SQ,” wherein some positions may be absent
  SITE (455)..(459): This region may encompass “SGGQQ,” “GAGQQ,” “GQGPY,” “AGQQ” or “SQ,” wherein some positions may be absent
  SITE (463)..(467): This region may encompass “SGGQQ,” “GAGQQ,” “GQGPY,” “AGQQ” or “SQ,” wherein some positions may be absent
  SITE (471)..(480): This region may encompass 6-10 residues
  SITE (484)..(547): This region may encompass 4-8 repeating “GPG-X1” repeating units, wherein X1 is “SGGQQ,” “GAGQQ,” “GQGPY,” “AGQQ” or “SQ,” and some positions may be absent
  SITE (487)..(491): This region may encompass “SGGQQ,” “GAGQQ,” “GQGPY,” “AGQQ” or “SQ,” wherein some positions may be absent
  SITE (495)..(499): This region may encompass “SGGQQ,” “GAGQQ,” “GQGPY,” “AGQQ” or “SQ,” wherein some positions may be absent
  SITE (503)..(507): This region may encompass “SGGQQ,” “GAGQQ,” “GQGPY,” “AGQQ” or “SQ,” wherein some positions may be absent
  SITE (511)..(515): This region may encompass “SGGQQ,” “GAGQQ,” “GQGPY,” “AGQQ” or “SQ,” wherein some positions may be absent
  SITE (519)..(523): This region may encompass “SGGQQ,” “GAGQQ,” “GQGPY,” “AGQQ” or “SQ,” wherein some positions may be absent
  SITE (527)..(531): This region may encompass “SGGQQ,” “GAGQQ,” “GQGPY,” “AGQQ” or “SQ,” wherein some positions may be absent
  SITE (535)..(539): This region may encompass “SGGQQ,” “GAGQQ,” “GQGPY,” “AGQQ” or “SQ,” wherein some positions may be absent
  SITE (543)..(547): This region may encompass “SGGQQ,” “GAGQQ,” “GQGPY,” “AGQQ” or “SQ,” wherein some positions may be absent
  SITE (551)..(560): This region may encompass 6-10 residues
  SITE (564)..(627): This region may encompass 4-8 repeating “GPG-X1” repeating units, wherein X1 is “SGGQQ,” “GAGQQ,” “GQGPY,” “AGQQ” or “SQ,” and some positions may be absent
  SITE (567)..(571): This region may encompass “SGGQQ,” “GAGQQ,” “GQGPY,” “AGQQ” or “SQ,” wherein some positions may be absent
  SITE (575)..(579): This region may encompass “SGGQQ,” “GAGQQ,” “GQGPY,” “AGQQ” or “SQ,” wherein some positions may be absent
  SITE (583)..(587): This region may encompass “SGGQQ,” “GAGQQ,” “GQGPY,” “AGQQ” or “SQ,” wherein some positions may be absent
  SITE (591)..(595): This region may encompass “SGGQQ,” “GAGQQ,” “GQGPY,” “AGQQ” or “SQ,” wherein some positions may be absent
  SITE (599)..(603): This region may encompass “SGGQQ,” “GAGQQ,” “GQGPY,” “AGQQ” or “SQ,” wherein some positions may be absent
  SITE (607)..(611): This region may encompass “SGGQQ,” “GAGQQ,” “GQGPY,” “AGQQ” or “SQ,” wherein some positions may be absent
  SITE (615)..(619): This region may encompass “SGGQQ,” “GAGQQ,” “GQGPY,” “AGQQ” or “SQ,” wherein some positions may be absent
  SITE (623)..(627): This region may encompass “SGGQQ,” “GAGQQ,” “GQGPY,” “AGQQ” or “SQ,” wherein some positions may be absent
  SITE (631)..(640): This region may encompass 6-10 residues
  SITE (644)..(707): This region may encompass 4-8 repeating “GPG-X1” repeating units, wherein X1 is “SGGQQ,” “GAGQQ,” “GQGPY,” “AGQQ” or “SQ,” and some positions may be absent
  SITE (647)..(651): This region may encompass “SGGQQ,” “GAGQQ,” “GQGPY,” “AGQQ” or “SQ,” wherein some positions may be absent
  SITE (655)..(659): This region may encompass “SGGQQ,” “GAGQQ,” “GQGPY,” “AGQQ” or “SQ,” wherein some positions may be absent
  SITE (663)..(667): This region may encompass “SGGQQ,” “GAGQQ,” “GQGPY,” “AGQQ” or “SQ,” wherein some positions may be absent
  SITE (671)..(675): This region may encompass “SGGQQ,” “GAGQQ,” “GQGPY,” “AGQQ” or “SQ,” wherein some positions may be absent
  SITE (679)..(683): This region may encompass “SGGQQ,” “GAGQQ,” “GQGPY,” “AGQQ” or “SQ,” wherein some positions may be absent
  SITE (687)..(691): This region may encompass “SGGQQ,” “GAGQQ,” “GQGPY,” “AGQQ” or “SQ,” wherein some positions may be absent
  SITE (695)..(699): This region may encompass “SGGQQ,” “GAGQQ,” “GQGPY,” “AGQQ” or “SQ,” wherein some positions may be absent
  SITE (703)..(707): This region may encompass “SGGQQ,” “GAGQQ,” “GQGPY,” “AGQQ” or “SQ,” wherein some positions may be absent
  SITE (711)..(720): This region may encompass 6-10 residues
  SITE (724)..(787): This region may encompass 4-8 repeating “GPG-X1” repeating units, wherein X1 is “SGGQQ,” “GAGQQ,” “GQGPY,” “AGQQ” or “SQ,” and some positions may be absent
  SITE (727)..(731): This region may encompass “SGGQQ,” “GAGQQ,” “GQGPY,” “AGQQ” or “SQ,” wherein some positions may be absent
  SITE (735)..(739): This region may encompass “SGGQQ,” “GAGQQ,” “GQGPY,” “AGQQ” or “SQ,” wherein some positions may be absent
  SITE (743)..(747): This region may encompass “SGGQQ,” “GAGQQ,” “GQGPY,” “AGQQ” or “SQ,” wherein some positions may be absent
  SITE (751)..(755): This region may encompass “SGGQQ,” “GAGQQ,” “GQGPY,” “AGQQ” or “SQ,” wherein some positions may be absent
  SITE (759)..(763): This region may encompass “SGGQQ,” “GAGQQ,” “GQGPY,” “AGQQ” or “SQ,” wherein some positions may be absent
  SITE (767)..(771): This region may encompass “SGGQQ,” “GAGQQ,” “GQGPY,” “AGQQ” or “SQ,” wherein some positions may be absent
  SITE (775)..(779): This region may encompass “SGGQQ,” “GAGQQ,” “GQGPY,” “AGQQ” or “SQ,” wherein some positions may be absent
  SITE (783)..(787): This region may encompass “SGGQQ,” “GAGQQ,” “GQGPY,” “AGQQ” or “SQ,” wherein some positions may be absent
  SITE (791)..(800): This region may encompass 6-10 residues
  SITE (804)..(867): This region may encompass 4-8 repeating “GPG-X1” repeating units, wherein X1 is “SGGQQ,” “GAGQQ,” “GQGPY,” “AGQQ” or “SQ,” and some positions may be absent
  SITE (807)..(811): This region may encompass “SGGQQ,” “GAGQQ,” “GQGPY,” “AGQQ” or “SQ,” wherein some positions may be absent
  SITE (815)..(819): This region may encompass “SGGQQ,” “GAGQQ,” “GQGPY,” “AGQQ” or “SQ,” wherein some positions may be absent
  SITE (823)..(827): This region may encompass “SGGQQ,” “GAGQQ,” “GQGPY,” “AGQQ” or “SQ,” wherein some positions may be absent
  SITE (831)..(835): This region may encompass “SGGQQ,” “GAGQQ,” “GQGPY,” “AGQQ” or “SQ,” wherein some positions may be absent
  SITE (839)..(843): This region may encompass “SGGQQ,” “GAGQQ,” “GQGPY,” “AGQQ” or “SQ,” wherein some positions may be absent
  SITE (847)..(851): This region may encompass “SGGQQ,” “GAGQQ,” “GQGPY,” “AGQQ” or “SQ,” wherein some positions may be absent
  SITE (855)..(859): This region may encompass “SGGQQ,” “GAGQQ,” “GQGPY,” “AGQQ” or “SQ,” wherein some positions may be absent
  SITE (863)..(867): This region may encompass “SGGQQ,” “GAGQQ,” “GQGPY,” “AGQQ” or “SQ,” wherein some positions may be absent
  SITE (871)..(880): This region may encompass 6-10 residues
  SITE (884)..(947): This region may encompass 4-8 repeating “GPG-X1” repeating units, wherein X1 is “SGGQQ,” “GAGQQ,” “GQGPY,” “AGQQ” or “SQ,” and some positions may be absent
  SITE (887)..(891): This region may encompass “SGGQQ,” “GAGQQ,” “GQGPY,” “AGQQ” or “SQ,” wherein some positions may be absent
  SITE (895)..(899): This region may encompass “SGGQQ,” “GAGQQ,” “GQGPY,” “AGQQ” or “SQ,” wherein some positions may be absent
  SITE (903)..(907): This region may encompass “SGGQQ,” “GAGQQ,” “GQGPY,” “AGQQ” or “SQ,” wherein some positions may be absent
  SITE (911)..(915): This region may encompass “SGGQQ,” “GAGQQ,” “GQGPY,” “AGQQ” or “SQ,” wherein some positions may be absent
  SITE (919)..(923): This region may encompass “SGGQQ,” “GAGQQ,” “GQGPY,” “AGQQ” or “SQ,” wherein some positions may be absent
  SITE (927)..(931): This region may encompass “SGGQQ,” “GAGQQ,” “GQGPY,” “AGQQ” or “SQ,” wherein some positions may be absent
  SITE (935)..(939): This region may encompass “SGGQQ,” “GAGQQ,” “GQGPY,” “AGQQ” or “SQ,” wherein some positions may be absent
  SITE (943)..(947): This region may encompass “SGGQQ,” “GAGQQ,” “GQGPY,” “AGQQ” or “SQ,” wherein some positions may be absent
  SITE (951)..(960): This region may encompass 6-10 residues
  SITE (964)..(1027): This region may encompass 4-8 repeating “GPG-X1” repeating units, wherein X1 is “SGGQQ,” “GAGQQ,” “GQGPY,” “AGQQ” or “SQ,” and some positions may be absent
  SITE (967)..(971): This region may encompass “SGGQQ,” “GAGQQ,” “GQGPY,” “AGQQ” or “SQ,” wherein some positions may be absent
  SITE (975)..(979): This region may encompass “SGGQQ,” “GAGQQ,” “GQGPY,” “AGQQ” or “SQ,” wherein some positions may be absent
  SITE (983)..(987): This region may encompass “SGGQQ,” “GAGQQ,” “GQGPY,” “AGQQ” or “SQ,” wherein some positions may be absent
  SITE (991)..(995): This region may encompass “SGGQQ,” “GAGQQ,” “GQGPY,” “AGQQ” or “SQ,” wherein some positions may be absent
  SITE (999)..(1003): This region may encompass “SGGQQ,” “GAGQQ,” “GQGPY,” “AGQQ” or “SQ,” wherein some positions may be absent
  SITE (1007)..(1011): This region may encompass “SGGQQ,” “GAGQQ,” “GQGPY,” “AGQQ” or “SQ,” wherein some positions may be absent
  SITE (1015)..(1019): This region may encompass “SGGQQ,” “GAGQQ,” “GQGPY,” “AGQQ” or “SQ,” wherein some positions may be absent
  SITE (1023)..(1027): This region may encompass “SGGQQ,” “GAGQQ,” “GQGPY,” “AGQQ” or “SQ,” wherein some positions may be absent
  SITE (1031)..(1040): This region may encompass 6-10 residues
  SITE (1044)..(1107): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1047)..(1051): This region may encompass “SGGQQ,” “GAGQQ,” “GQGPY,” “AGQQ” or “SQ,” wherein some positions may be absent
  SITE (1055)..(1059): This region may encompass “SGGQQ,” “GAGQQ,” “GQGPY,” “AGQQ” or “SQ,” wherein some positions may be absent
  SITE (1063)..(1067): This region may encompass “SGGQQ,” “GAGQQ,” “GQGPY,” “AGQQ” or “SQ,” wherein some positions may be absent
  SITE (1071)..(1075): This region may encompass “SGGQQ,” “GAGQQ,” “GQGPY,” “AGQQ” or “SQ,” wherein some positions may be absent
  SITE (1079)..(1083): This region may encompass “SGGQQ,” “GAGQQ,” “GQGPY,” “AGQQ” or “SQ,” wherein some positions may be absent
  SITE (1087)..(1091): This region may encompass “SGGQQ,” “GAGQQ,” “GQGPY,” “AGQQ” or “SQ,” wherein some positions may be absent
  SITE (1095)..(1099): This region may encompass “SGGQQ,” “GAGQQ,” “GQGPY,” “AGQQ” or “SQ,” wherein some positions may be absent
  SITE (1103)..(1107): This region may encompass “SGGQQ,” “GAGQQ,” “GQGPY,” “AGQQ” or “SQ,” wherein some positions may be absent
  SITE (1111)..(1120): This region may encompass 6-10 residues
  SITE (1124)..(1187): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1127)..(1131): This region may encompass “SGGQQ,” “GAGQQ,” “GQGPY,” “AGQQ” or “SQ,” wherein some positions may be absent
  SITE (1135)..(1139): This region may encompass “SGGQQ,” “GAGQQ,” “GQGPY,” “AGQQ” or “SQ,” wherein some positions may be absent
  SITE (1143)..(1147): This region may encompass “SGGQQ,” “GAGQQ,” “GQGPY,” “AGQQ” or “SQ,” wherein some positions may be absent
  SITE (1151)..(1155): This region may encompass “SGGQQ,” “GAGQQ,” “GQGPY,” “AGQQ” or “SQ,” wherein some positions may be absent
  SITE (1159)..(1163): This region may encompass “SGGQQ,” “GAGQQ,” “GQGPY,” “AGQQ” or “SQ,” wherein some positions may be absent
  SITE (1167)..(1171): This region may encompass “SGGQQ,” “GAGQQ,” “GQGPY,” “AGQQ” or “SQ,” wherein some positions may be absent
  SITE (1175)..(1179): This region may encompass “SGGQQ,” “GAGQQ,” “GQGPY,” “AGQQ” or “SQ,” wherein some positions may be absent
  SITE (1183)..(1187): This region may encompass “SGGQQ,” “GAGQQ,” “GQGPY,” “AGQQ” or “SQ,” wherein some positions may be absent
  SITE (1191)..(1200): This region may encompass 6-10 residues
  SITE (1204)..(1267): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1207)..(1211): This region may encompass “SGGQQ,” “GAGQQ,” “GQGPY,” “AGQQ” or “SQ,” wherein some positions may be absent
  SITE (1215)..(1219): This region may encompass “SGGQQ,” “GAGQQ,” “GQGPY,” “AGQQ” or “SQ,” wherein some positions may be absent
  SITE (1223)..(1227): This region may encompass “SGGQQ,” “GAGQQ,” “GQGPY,” “AGQQ” or “SQ,” wherein some positions may be absent
  SITE (1231)..(1235): This region may encompass “SGGQQ,” “GAGQQ,” “GQGPY,” “AGQQ” or “SQ,” wherein some positions may be absent
  SITE (1239)..(1243): This region may encompass “SGGQQ,” “GAGQQ,” “GQGPY,” “AGQQ” or “SQ,” wherein some positions may be absent
  SITE (1247)..(1251): This region may encompass “SGGQQ,” “GAGQQ,” “GQGPY,” “AGQQ” or “SQ,” wherein some positions may be absent
  SITE (1255)..(1259): This region may encompass “SGGQQ,” “GAGQQ,” “GQGPY,” “AGQQ” or “SQ,” wherein some positions may be absent
  SITE (1263)..(1267): This region may encompass “SGGQQ,” “GAGQQ,” “GQGPY,” “AGQQ” or “SQ,” wherein some positions may be absent
  SITE (1271)..(1280): This region may encompass 6-10 residues
  SITE (1284)..(1347): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1287)..(1291): This region may encompass “SGGQQ,” “GAGQQ,” “GQGPY,” “AGQQ” or “SQ,” wherein some positions may be absent
  SITE (1295)..(1299): This region may encompass “SGGQQ,” “GAGQQ,” “GQGPY,” “AGQQ” or “SQ,” wherein some positions may be absent
  SITE (1303)..(1307): This region may encompass “SGGQQ,” “GAGQQ,” “GQGPY,” “AGQQ” or “SQ,” wherein some positions may be absent
  SITE (1311)..(1315): This region may encompass “SGGQQ,” “GAGQQ,” “GQGPY,” “AGQQ” or “SQ,” wherein some positions may be absent
  SITE (1319)..(1323): This region may encompass “SGGQQ,” “GAGQQ,” “GQGPY,” “AGQQ” or “SQ,” wherein some positions may be absent
  SITE (1327)..(1331): This region may encompass “SGGQQ,” “GAGQQ,” “GQGPY,” “AGQQ” or “SQ,” wherein some positions may be absent
  SITE (1335)..(1339): This region may encompass “SGGQQ,” “GAGQQ,” “GQGPY,” “AGQQ” or “SQ,” wherein some positions may be absent
  SITE (1343)..(1347): This region may encompass “SGGQQ,” “GAGQQ,” “GQGPY,” “AGQQ” or “SQ,” wherein some positions may be absent
  SITE (1351)..(1360): This region may encompass 6-10 residues
  SITE (1364)..(1427): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1367)..(1371): This region may encompass “SGGQQ,” “GAGQQ,” “GQGPY,” “AGQQ” or “SQ,” wherein some positions may be absent
  SITE (1375)..(1379): This region may encompass “SGGQQ,” “GAGQQ,” “GQGPY,” “AGQQ” or “SQ,” wherein some positions may be absent
  SITE (1383)..(1387): This region may encompass “SGGQQ,” “GAGQQ,” “GQGPY,” “AGQQ” or “SQ,” wherein some positions may be absent
  SITE (1391)..(1395): This region may encompass “SGGQQ,” “GAGQQ,” “GQGPY,” “AGQQ” or “SQ,” wherein some positions may be absent
  SITE (1399)..(1403): This region may encompass “SGGQQ,” “GAGQQ,” “GQGPY,” “AGQQ” or “SQ,” wherein some positions may be absent
  SITE (1407)..(1411): This region may encompass “SGGQQ,” “GAGQQ,” “GQGPY,” “AGQQ” or “SQ,” wherein some positions may be absent
  SITE (1415)..(1419): This region may encompass “SGGQQ,” “GAGQQ,” “GQGPY,” “AGQQ” or “SQ,” wherein some positions may be absent
  SITE (1423)..(1427): This region may encompass “SGGQQ,” “GAGQQ,” “GQGPY,” “AGQQ” or “SQ,” wherein some positions may be absent
  SITE (1431)..(1440): This region may encompass 6-10 residues
  SITE (1444)..(1507): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1447)..(1451): This region may encompass “SGGQQ,” “GAGQQ,” “GQGPY,” “AGQQ” or “SQ,” wherein some positions may be absent
  SITE (1455)..(1459): This region may encompass “SGGQQ,” “GAGQQ,” “GQGPY,” “AGQQ” or “SQ,” wherein some positions may be absent
  SITE (1463)..(1467): This region may encompass “SGGQQ,” “GAGQQ,” “GQGPY,” “AGQQ” or “SQ,” wherein some positions may be absent
  SITE (1471)..(1475): This region may encompass “SGGQQ,” “GAGQQ,” “GQGPY,” “AGQQ” or “SQ,” wherein some positions may be absent
  SITE (1479)..(1483): This region may encompass “SGGQQ,” “GAGQQ,” “GQGPY,” “AGQQ” or “SQ,” wherein some positions may be absent
  SITE (1487)..(1491): This region may encompass “SGGQQ,” “GAGQQ,” “GQGPY,” “AGQQ” or “SQ,” wherein some positions may be absent
  SITE (1495)..(1499): This region may encompass “SGGQQ,” “GAGQQ,” “GQGPY,” “AGQQ” or “SQ,” wherein some positions may be absent
  SITE (1503)..(1507): This region may encompass “SGGQQ,” “GAGQQ,” “GQGPY,” “AGQQ” or “SQ,” wherein some positions may be absent
  SITE (1511)..(1520): This region may encompass 6-10 residues
  SITE (1524)..(1587): This region may encompass 4-8 repeating “GPG-X1” repeating units, wherein X1 is “SGGQQ,” “GAGQQ,” “GQGPY,” “AGQQ” or “SQ,” and some positions may be absent
  SITE (1527)..(1531): This region may encompass “SGGQQ,” “GAGQQ,” “GQGPY,” “AGQQ” or “SQ,” wherein some positions may be absent
  SITE (1535)..(1539): This region may encompass “SGGQQ,” “GAGQQ,” “GQGPY,” “AGQQ” or “SQ,” wherein some positions may be absent
  SITE (1543)..(1547): This region may encompass “SGGQQ,” “GAGQQ,” “GQGPY,” “AGQQ” or “SQ,” wherein some positions may be absent
  SITE (1551)..(1555): This region may encompass “SGGQQ,” “GAGQQ,” “GQGPY,” “AGQQ” or “SQ,” wherein some positions may be absent
  SITE (1559)..(1563): This region may encompass “SGGQQ,” “GAGQQ,” “GQGPY,” “AGQQ” or “SQ,” wherein some positions may be absent
  SITE (1567)..(1571): This region may encompass “SGGQQ,” “GAGQQ,” “GQGPY,” “AGQQ” or “SQ,” wherein some positions may be absent
  SITE (1575)..(1579): This region may encompass “SGGQQ,” “GAGQQ,” “GQGPY,” “AGQQ” or “SQ,” wherein some positions may be absent
  SITE (1583)..(1587): This region may encompass “SGGQQ,” “GAGQQ,” “GQGPY,” “AGQQ” or “SQ,” wherein some positions may be absent
  SITE (1591)..(1600): This region may encompass 6-10 residues
GGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAAGGYGPGXXXXXGPGXXXXXGPGXXXXXGPGXXXXXGPGXXXXXGPGXXXXXGPGXXXXXGPGXXXXXGPSAAAAAAAAAA

SEQ ID NO: 35 - protein, 5 aa, Artificial Sequence
  Description of Artificial Sequence Synthetic peptide
SGGQQ

SEQ ID NO: 36 - protein, 5 aa, Artificial Sequence
  Description of Artificial Sequence Synthetic peptide
GAGQQ

SEQ ID NO: 37 - protein, 5 aa, Artificial Sequence
  Description of Artificial Sequence Synthetic peptide
GQGPY

SEQ ID NO: 38 - protein, 4 aa, Artificial Sequence
  Description of Artificial Sequence Synthetic peptide
AGQQ